(19) United States
(12) Patent Application Publication
(10) Pub. No.: US 2013/0039990 A1
(43) Pub. Date: Feb. 14, 2013

(54) INJECTABLE, LOAD-BEARING CELL/MICROBEAD/CALCIUM PHOSPHATE BONE PASTE FOR BONE TISSUE ENGINEERING

(71) Applicants: Huakun Xu, Frederick, MD (US); Michael Weir, Silver Spring, MD (US)

(72) Inventors: Huakun Xu, Frederick, MD (US); Michael Weir, Silver Spring, MD (US)

(73) Assignee: UNIVERSITY OF MARYLAND, BALTIMORE, Baltimore, MD (US)

(21) Appl. No.: 13/695,122

(22) PCT Filed: Apr. 29, 2011
(86) PCT No.: PCT/US2011/034457

§ 371 Date: Oct. 29, 2012

Related U.S. Application Data
(60) Provisional application No. 61/329,814, filed on Apr. 30, 2010.

(51) Int. Cl. A61K 9/50; A61K 35/32; A61K 35/44; A61K 9/14; A61K 35/54; A61P 19/08; A61K 47/42; A61K 35/12; A61K 35/28

(52) U.S. Cl. 424/492; 424/490; 424/493; 424/497; 424/93.7; 424/491; 514/773; 424/400

(57) ABSTRACT

The invention provides injectable, stem cell-containing calcium phosphate bone pastes for bone tissue engineering and methods of making and using the same.

### STATEMENT OF GOVERNMENT SUPPORT

This invention was made with government support under R01Grant Nos. DE014190 and DE017974 awarded by the National Institutes of Health. The government has certain rights in the invention.

### FIELD

The invention provides injectable, cell-containing calcium phosphate bone pastes for bone tissue engineering, and methods of making and using the same.

### BACKGROUND

Bone transplantation is used to treat bone defects arising from trauma, disease, congenital deformity, or tumor resection. Bone is the second most transplanted tissue after blood; however, current bone transplantation methods involve certain disadvantages. For example, bone autografts (i.e., bone harvested from a patient to be treated), are of limited availability and incur donor site morbidity. Bone allografts (i.e., bone harvested from a person who is not the patient to be treated), carry a risk of disease transmission. With seven million bone fractures per year in the U.S., and musculoskeletal conditions costing $215 billion [1,2], new stem cell-biomaterial treatments are needed. Stem cell-based tissue engineering has immense potential to regenerate damaged and diseased tissues [3-7].

Human bone marrow mesenchymal stem cells (hBMSCs) can differentiate into osteoblasts, adipocytes, chondrocytes, myoblasts, neurons, and fibroblasts [8-10]. hBMSCs can be harvested from a patient, expanded in culture, induced to differentiate, and combined with a scaffold to repair bone defects. However, harvesting of autogenous hBMSCs requires an invasive procedure. Moreover, autogenous hBMSCs display lower self-renewal potential with aging of the individual from whom the cells are obtained.

Human umbilical cord mesenchymal stem cells (hUCMSCs) have been used in tissue engineering [11-16]. Umbilical cords can provide an inexpensive and inexhaustible stem cell source, without the invasive procedure of hBMSCs, and without the controversies of embryonic stem cells (hESCs). hUCMSCs are primitive MSCs that exhibit a high plasticity and developmental flexibility and appear to cause no immunorejection in vivo [12]. hUCMSCs have been cultured on tissue culture plastic [13], polymer scaffolds [16], and calcium phosphate scaffolds for tissue engineering [17-19].

Calcium phosphate (CaP) scaffolds are important for bone repair because they are bioactive, mimic the bone minerals, and can bond to neighboring bone, in contrast to bioinert implants that can form undesirable fibrous capsules [20-22]. The CaP minerals provide a preferred substrate for cell attachment and expression of the osteoblast phenotype [23,24]. However, for pre-formed bioceramic scaffolds to fit into a bone cavity, a surgeon must machine the graft or carve the surgical site, leading to increases in bone loss, trauma, and surgical time [2]. Pre-formed scaffolds have other drawbacks, including the difficulty in seeding cells deeply into the scaffold and the inability to inject such scaffolds in minimally-invasive surgeries [2,10].

Injectable scaffolds for cell delivery are advantageous because they can: (i) shorten the surgical operation time; (ii) minimize the damaging effects of large muscle retraction; (iii) reduce postoperative pain and scar size; (iv) achieve rapid recovery; and (v) reduce cost. Various injectable hydrogel and polymer carriers can be used for stem cell delivery [10,25]. However, current injectable carriers cannot be used in load-bearing repairs [10,25], such as those required in bone. For example, hydrogel scaffolds do not possess the mechanical strength to be used in load bearing applications [25].

Mechanical properties of scaffolding materials are of crucial importance in regeneration of load-bearing tissues such as bone. Specifically, scaffolding materials must be able to withstand stresses to avoid scaffold fracture and to maintain scaffold structure to define the shape of the regenerated tissue. However, to date, an injectable, bioactive, and strong scaffold for stem cell encapsulation and bone engineering has not yet been developed.

Hydroxyapatite (HA) and other calcium phosphate (CaP) bioceramics are useful in hard tissue repair because of their excellent biocompatibility [5,8,10,20-24]. When implanted into an osseous site, bone bioactive materials such as HA and other CaP implants and coatings provide an ideal environment for cellular reaction and colonization by osteoblasts. This leads to a tissue response termed osteoconduction in which bone grows on and bonds to the implant, promoting a functional interface. These bioceramics are highly useful for bone repair. One drawback is that sintered HA implants are generally not resorbable. Another limitation is that these bioceramics are pre-forms that require machining and may leave gaps when fitted into a bone cavity.

In contrast to CaP bioceramics, calcium phosphate cements can self-set in the bone site with intimate adaptation to complex shapes, can be easily contoured for esthetics in craniofacial repairs, and they are highly osteoconductive and bioresorbable [26-32]. Calcium phosphate cements (CPCs) can be injected or molded, and set in situ to form a bioactive scaffold that bonds to bone [26-29]. The first CPC was approved by the Food and Drug Administration (FDA) in 1996 for craniofacial repairs [26, 30-32]. CPC has excellent osteoconductivity and can be replaced by new bone [30-32]. In previous studies, alginate hydrogel beads [19,33,34] and tubular hydrogels [35] have been used to encapsulate cells in CPC. The hydrogel protects the cells during the CPC mixing and setting reaction. Once CPC has set, the hydrogel dissolves and releases the cells throughout the entire CPC implant, while concomitantly creating macroporosity. However, previously described hydrogel beads have diameters of 2-3 mm. Pastes using such large hydrogel beads are not suitable for injection, as would be desirable, e.g., in minimally invasive surgeries.

In view of the foregoing, there is a need for injectable materials that can be used to repair load-bearing bone.

### SUMMARY

Provided herein are the first injectable, self-setting, bioactive, and mechanically-strong cell-containing bone pastes for bone tissue engineering. The new bone pastes employ a calcium phosphate cement containing cell-encapuslating microbeads and, optionally, additional components, such as chitosan and/or fibers for reinforcement. In particular aspects, the cells are stem cells.

Also provided herein are novel alginate-fibrin microbeads that can be used in the bone pastes of the present invention, as well as other applications.

In particular, and in a first aspect, described herein is a bone paste comprising a calcium phosphate cement and microbeads, wherein the microbeads encapsulate cells.

The microbeads of the bone paste can include hydrogel microbeads, for example, but not limited to, microbeads comprising alginate, partially oxidized alginate, oxidized alginate, alginate-fibrin, partially oxidized alginate-fibrin, oxidized alginate-fibrin, poly(ethylene glycol diacrylate), poly(ethylene glycol)-anhydride dimethacrylate, gelatin, chemically cross-linked polymers, ionically cross-linked polymers, heat-polymerized polymers, or photopolymerized polymers. In one embodiment, the microbeads are alginate-fibrin microbeads. When the microbeads are alginate-fibrin microbeads they may comprise a fibrinogen mass fraction of from about 0.05% to about 1%, such as, but not limited to, a fibrinogen mass fraction of about 0.12%.

The microbeads of the bone paste may be present in a volume of about 40 to 60%, and they may have an average diameter of less than about 2 millimeters.

The cells of the bone paste may be stem cells, for example, but not limited to, one or more of human umbilical cord mesenchymal stem cells, bone marrow stem cells, embryonic stem cells, pluripotent stem cells, induced pluripotent stem cells, multipotent stem cells, progenitor cells, and osteoblasts.

The calcium phosphate cement of the bone paste may comprise, for example, but not be limited to, one or more ingredients selected from the group consisting of tetracalcium phosphate (TTCP) (Ca₄(PO₄)₂O), dicalcium phosphate anhydrous (DCPA) (CaHPO₄), dicalcium phosphate dihydrate (CaHPO₄.2H₂O), tricalcium phosphate (Ca₃[PO₄]₂), α-tricalcium phosphate (α-Ca₃(PO₄)₂), β-tricalcium phosphate (β-Ca₃(PO₄)₂), octacalcium phosphate (Ca₈H₂(PO₄)₆.5H₂O), amorphous calcium phosphate (Ca₃(PO₄)₂), calcium carbonate (CaCO₃), calcium hydroxide (Ca[OH]₂), and hydroxyapatite (Ca₁₀(PO₄)₆(OH)₂), and mixtures thereof.

In one embodiment, the calcium phosphate cement of the bone paste comprises tetracalcium phosphate and dicalcium phosphate anhydrous. In this embodiment, the calcium phosphate cement may comprise, for example, but not be limited to, a molar ratio of tetracalcium phosphate to dicalcium phosphate anhydrous of about 1:5 to about 5:1, a molar ratio of tetracalcium phosphate to dicalcium phosphate anhydrous of about 1:3 to about 1:1, or the calcium phosphate cement comprises an approximately 1:1 molar ratio of tetracalcium phosphate to dicalcium phosphate anhydrous.

The calcium phosphate cement of the bone paste may further comprise chitosan.

The calcium phosphate cement of the bone paste may further comprise fibers. The fibers may be, but are not limited to, degradable fibers.

The calcium phosphate cement of the bone paste may further comprise a porogen. As an example, but not a limitation, the porogen may be NaHCO₃ and citric acid. When the porogen is NaHCO₃ and citric acid, the mass fraction of NaHCO₃ may be from about 5% to about 30%, and the mass fraction of citric acid may be from about 50% to about 60%.

The bone paste may further comprise a bioactive agent. The bone paste may also be injectable.

In a second aspect, described herein is a bone paste, comprising: (a) calcium phosphate cement, wherein the calcium phosphate cement comprises a porogen; (b) alginate-fibrin microbeads, wherein the microbeads encapsulate cells; (c) chitosan; and (d) degradable fibers.

The alginate-fibrin microbeads of the bone paste may comprise a fibrinogen mass fraction of from about 0.05% to about 1%, for example, but not limited to, a fibrinogen mass fraction of about 0.12%. The microbeads may be present in a volume of about 40 to 60%, and they may have an average diameter of less than about 2 millimeters.

The cells of the bone paste may be stem cells, for example, but not limited to, one or more of human umbilical cord mesenchymal stem cells, bone marrow stem cells, embryonic stem cells, pluripotent stem cells, induced pluripotent stem cells, multipotent stem cells, progenitor cells, and osteoblasts.

The calcium phosphate cement of the bone paste may comprise, for example, but not be limited to, one or more ingredients selected from the group consisting of tetracalcium phosphate (TTCP) (Ca₄(PO₄)₂O), dicalcium phosphate anhydrous (DCPA) (CaHPO₄), dicalcium phosphate dihydrate (CaHPO₄.2H₂O), tricalcium phosphate (Ca₃[PO₄]₂), α-tricalcium phosphate (α-Ca₃(PO₄)₂), β-tricalcium phosphate (β-Ca₃(PO₄)₂), octacalcium phosphate (Ca₈H₂(PO₄)₆.5H₂O), amorphous calcium phosphate (Ca₃(PO₄)₂), calcium carbonate (CaCO₃), calcium hydroxide (Ca[OH]₂), and hydroxyapatite (Ca₁₀(PO₄)₆(OH)₂), and mixtures thereof.

In one embodiment, the calcium phosphate cement of the bone paste comprises tetracalcium phosphate and dicalcium phosphate anhydrous. In this embodiment, the calcium phosphate cement may comprise, for example, but not be limited to, a molar ratio of tetracalcium phosphate to dicalcium phosphate anhydrous of about 1:5 to about 5:1, a molar ratio of tetracalcium phosphate to dicalcium phosphate anhydrous of about 1:3 to about 1:1, or the calcium phosphate cement comprises an approximately 1:1 molar ratio of tetracalcium phosphate to dicalcium phosphate anhydrous.

The porogen of the bone paste may be, but is not limited to, NaHCO₃ and citric acid. When the porogen is NaHCO₃ and citric acid, the mass fractions of NaHCO₃ may be from about 5% to about 30%, and the mass fraction of citric acid may be from about 50% to about 60%.

The bone paste may further comprise a bioactive agent. The bone paste may also be injectable.

The bone paste may have the characteristic, but is not required to do so, that at least 90% of a sample of the bone paste can be injected using an injection force of about 100 Newtons or less.

The bone paste may have the characteristic, but is not required to do so, that a viability of the cells after injection of the bone paste is at least about 65% of the viability of cells in non-injected bone paste.

The bone paste may have the characteristic, but is not required to do so, that the bone paste, after hardening, has a flexural strength substantially similar to that of natural cancellous bone.

In a third aspect, described herein is a method of repairing or remodeling a bone, comprising administering to a bone an effective amount of any of the bone pastes described herein and allowing the bone paste to harden, thereby repairing or remodeling the bone.

In a fourth aspect, described herein is an alginate-fibrin microbead, wherein said microbead comprises alginate and fibrinogen. The alginate-fibrin microbead may comprise a fibrinogen mass fraction of from about 0.05% to about 1%, for example, but not limited to, a fibrinogen mass fraction of about 0.12%. In one embodiment, the alginate-fibrin microbead has an average diameter of less than about 2 millimeters.

### BRIEF DESCRIPTION OF THE DRAWINGS

FIG. 1 shows stem cell-encapsulating microbead synthesis and mechanical properties of stem cell constructs. (A) Schematic of the hUCMSC-encapsulating alginate hydrogel microbead synthesizer. (B) Histogram of the hUCMSC-encapsulating hydrogel microbead size. (C) Flexural strength, (D) elastic modulus, and (E) work-of-fracture (toughness) of CPC constructs containing 50 vol % of hUCMSC-encapsulating beads (mean±sd; n=5). The large beads were the same as those in previous studies [19,33,34]. The microbeads were fabricated in the present study. Using the new microbeads in CPC greatly increased the load-bearing capability of the stem cell-CPC composite.

FIG. 2 shows injectability of hUCMSC—CPC composites (mean±sd; n=3). (A) Percentage of paste extruded. (B) Maximum injection force recorded during injection. All the pastes were nearly completely extruded at relatively small injection forces. Chitosan had a cohesive and lubricating effect on the TTCP and DCPA particles, thereby lowering the injection force. The CPC-chitosan-fiber-microbeads paste was as injectable as the CPC-microbeads paste. The hUCMSC-encapsulating microbeads volume fraction in the paste was 50%. The fiber length was 3 mm, and the fiber volume fraction was 20%.

FIG. 3 shows mechanical properties of injectable stem cell composites. Each specimen contained 50 vol % of microbeads with 150,000 hUCMSCs. Strength and work-of-fracture (mean±sd; n=5) were greatly increased with the incorporation of chitosan and chopped fibers. Values for CPC-microbeads, CPC-chitosan-microbeads, and CPC-chitosan-fiber-microbeads were measured in the present study. Values for cancellous bone, injectable polymeric carrier and hydrogels are from the literature as described herein.

FIG. 4 shows hUCMSC viability without injection and after injection. (A) hUCMSCs in hydrogel microbeads alone (without CPC and without injection). (B) hUCMSCs in microbeads after mixing with CPC-chitosan-fiber paste and after injection. Both were cultured for 1d. Live cells were stained green and were numerous. Dead cells were stained red and were few (example in C). (D) Percentage of live cells (mean±sd; n=5). (E) Live cell density. Paste mixing and injection did not harm the encapsulated hUCMSCs.

FIG. 5 shows effects of injectable composite composition and culture time on hUCMSCs. For the percentage of live cells or cell density (mean±sd; n=5), all the values were statistically similar, indicating that: (i) encapsulation in CPC did not compromise the cell viability; (ii) using chitosan and fibers for reinforcement did not adversely affect the cell viability; (iii) the hUCMSCs remained viable during encapsulation for 14 d.

FIG. 6 shows RT-PCR results of alkaline phosphatase (ALP) and osteocalcin (OC) gene expressions during osteogenic differentiation of the encapsulated hUCMSCs (mean±sd; n=5). ALP and OC were greatly increased at 7 d. Encapsulation in CPC did not adversely affect the ALP or OC, and adding chitosan and fibers for reinforcement did not compromise the ALP or OC, compared to hUCMSCs in hydrogel microbeads alone.

FIG. 7 shows colorimetric pNPP assay of ALP protein synthesis by the encapsulated hUCMSCs. The ALP enzyme production greatly increased at 14 d over that at 7 d, and then decreased at 21d (mean±sd; n=5). ALP activity in all the injectable CPC-based composites matched that in hydrogel microbeads without CPC.

FIG. 8 shows synthesis of bone minerals by the encapsulated hUCMSCs. (A-D) Typical mineral staining photos. Minerals in the harvested microbeads emitted red fluorescence when stained with xylenol orange. The mineralization increased rapidly with time. This trend was the same in all four constructs. (E) Mineral area fraction (mean±sd; n=5). Mineral area at 7 d was 50-fold higher than that at 1d. Mineral area at 14 d was nearly 2-fold higher than that at 7 d. hUCMSCs encapsulated in all three injectable CPC-based composites matched the mineralization in hydrogel microbeads without CPC.

FIG. 9 shows optical photos of hUCMSC-encapsulating alginate microbeads (type 1) cultured for (A) 1d, (B) 7 d, (C) 14d, and (D) 21d. A blue filter was used to enhance the contrast of the microbeads. The long arrow in (A) indicates the boundary of a relatively large microbead. Most of the microbeads appeared nearly spherical. The long arrow in (B) indicates cells inside a microbead. The short arrows in (B) and (D) indicate examples of smaller microbeads. (E-H) Typical fluorescent images of live/dead staining at 1, 7, 14 and 21d, respectively. Five samples were cultured at each time point (n=5).

FIG. 10 shows optical photos of hUCMSC-encapsulating oxidized alginate microbeads (type 2) at (A) 1d, (B) 7 d, (C) 14 d, and (D) 21d. The microbeads started to degrade at 14 d. At 21d, some microbeads started to fall apart (arrows in D). (E-H) Fluorescent images of live/dead staining at 1, 7, 14 and 21d, respectively. The live cells appeared as small green dots at 1 and 7 d. Many cells still appeared as green dots at 14 and 21d. However, there were cells with a spreading morphology at 14 and 21d, indicative of cell release from the microbeads and attaching to the tissue culture polystyrene.

FIG. 11 shows optical photos of hUCMSC-encapsulating oxidized alginate-fibrin microbeads (type 3) at (A) 1d with low magnification, (B) 1d with higher magnification, (C), 4 d, (D) 7 d, (E) 14 d, and (F) 21d. The microbeads were intact and did not degrade at 1d. At 4 d, they started to degrade, with arrows in (C) indicating the breakdown of the microbead boundary and the release of the encapsulated hUCMSCs. The degradation accelerated at 7 d and numerous cells were released (arrows in D). The microbeads fell apart in (E) and became small pieces in (F).

FIG. 12 shows typical fluorescent live/dead staining images for the oxidized alginate-fibrin microbeads (type 3). Live cells were stained green at (A) 1d, (B) 4 d, (C) 7 d, (D) 14 d, (E) 21d. (F) Dead cells were stained red at 21d. At 1d, the cells appeared as green dots. At 4 d, some cells were released from the microbeads and showed a spreading morphology. At 7 d, the number of released cells greatly increased and attached on the tissue culture polystyrene, showing a healthy polygonal morphology. The released cells proliferated, forming a confluent monolayer at 21d. Dead cells were few from 1d to 21d.

FIG. 13 shows hUCMSC viability: (A) percentage of live cells, and (B) live cell density. Each value is the mean of five measurements, with the error bar showing one standard deviation (mean±sd; n=5).

FIG. 14 shows RT-PCR results for the oxidized alginate-fibrin group. Each value is mean±sd; n=5. hUCMSCs cultured for 1d in control media without the osteogenic supplements served as control and its value was set as being 1. When cultured in osteogenic media, the ALP gene expression peaked at 7 d. Runx2 peaked at 14 d. OC and collagen I peaked at 21d. Therefore, the hUCMSCs released from the oxidized alginate-fibrin microbeads had differentiated into the osteogenic lineage.

FIG. 15 shows mineralization of hUMSCs released from the oxidized alginate-fibrin microbeads at: (A) 7 d, (B) 14 d, and (C) 21d. Culture was performed in osteogenic media. The ARS stained the calcium minerals into a red color. (D) The mineral concentration was measured by an osteogenesis assay (n=5). These results demonstrate that the hUCMSCs released from the degradable microbeads successfully synthesized bone minerals.

FIG. 16 shows (A) schematic of hUCMSC-encapsulating hydrogel microbeads inside CPC scaffold immersed in culture media in a culture well. (B) and (C) Effects of gas-foam porogen mass fraction in CPC on the percentage of live cells and live cell density, respectively. Each value is the mean of five measurements, with the error showing one standard deviation (mean±sd; n=5).

FIG. 17 shows live/dead stained photos of cells inside CPC. Live cells were stained green and dead cells were stained red. Labels on the left side indicate the porogen mass fraction in CPC. The number of live cells increased, and dead cells decreased, when the porogen was increased from 0% to 10%, 15% and 20%.

FIG. 18 shows effect of culture time from 1d to 21d on the encapsulated hUCMSCs inside CPC scaffold. Each CPC construct contained the same gas-foam porogen mass fraction of 15%. (A) Percentage of live cells, and (B) live cell density. There was a significant decrease in the percentage of live cells in the first week. However, cell proliferation significantly increased the percentage of live cells as well as the live cell density over time. Each value is mean±sd; n=5.

FIG. 19 shows hUCMSC release from degradable alginate-fibrin microbeads inside CPC. The bottom CPC surface as shown in FIG. 19A was live/dead stained and examined. (A) At 7 d, some cells showed as green dots, indicating that they were still encapsulated in fragments of the alginate-fibrin microbeads. Other cells had a spread and spindle morphology indicating that they had been released from the microbeads and attached to CPC. (B) The spread and elongated cell morphology at 14 d at a higher magnification. (C) At 21d, the number of released and attached cells greatly increased. (D) For comparison, alginate microbeads without fibrin, similar to those of previous studies, did not degrade and did not release the cells. Cells remained as green dots and did not spread, even at 21d.

FIG. 20 shows SEM micrograph of CPC porosity at (A) 0%, (B) 10%, and (C) 20% foam porogen. An example of macropore interconnection is indicated by the long arrow in (B). Openings inside macropores are indicated by the short arrows. Numerous micropores in the CPC matrix are shown at a higher magnification in (C).

FIG. 21 shows effect of gas-foaming porogen mass fraction on: (A) the density, and (B) the porosity of CPC. The purpose was to measure the porosity in the CPC matrix, hence the specimens contained no fibers. When the foam agent was increased from 0% to 20%, the pore volume fraction in CPC increased from 46.8% (intrinsic microporosity in CPC) to 78.4% (microporosity+macroporosity). Each value is mean±sd; n=5.

FIG. 22 shows effect of foam porogen content on CPC mechanical properties: (A) Flexural strength, (B) work-of-fracture, and (C) elastic modulus. For the group with fibers, suture fibers at a length of 3 mm and a volume fraction of 20% were used in CPC. Each value is mean±sd; n=5. Fiber reinforcement greatly increased the mechanical properties of gas-foaming CPC scaffold.

### DETAILED DESCRIPTION

The need for bone regeneration or remodeling can arise from trauma, disease, congenital deformity, or tumor resection. Stem cell-scaffold approaches hold immense promise for bone tissue engineering. Currently, pre-formed scaffolds for cell delivery have drawbacks, including the difficulty of seeding cells deeply into the scaffold and the inability to inject such scaffolds in repair procedures involving minimally invasive surgeries. Current injectable polymeric carriers and hydrogels are too weak for load-bearing orthopedic application.

Provided herein is the first injectable, strong, and bioactive cell-containing bone paste for bone tissue engineering. In the bone pastes described herein, a calcium phosphate cement (CPC) is combined with polymer (e.g., hydrogel) microbeads encapsulating cells, such as human umbilical cord mesenchymal stem cells (hUCMSCs). The CPC may optionally contain chitosan and/or degradable fibers for reinforcement. The CPC may further optionally contain a porogen to increase the porosity of the bone paste. The resulting cell-encapsulating bone paste is fully injectable under small injection forces. The cell viability post-injection matches that in hydrogel without CPC and without injection. Stem cells in the injectable bone pastes maintain their ability to osteodifferentiate, as indicated by expression of osteogenic markers such as alkaline phosphatase and osteocalcin and by accumulation of bone minerals. Mechanical properties of the bone pastes match the reported values of cancellous bone, and are much higher than previous injectable polymeric and hydrogel carriers.

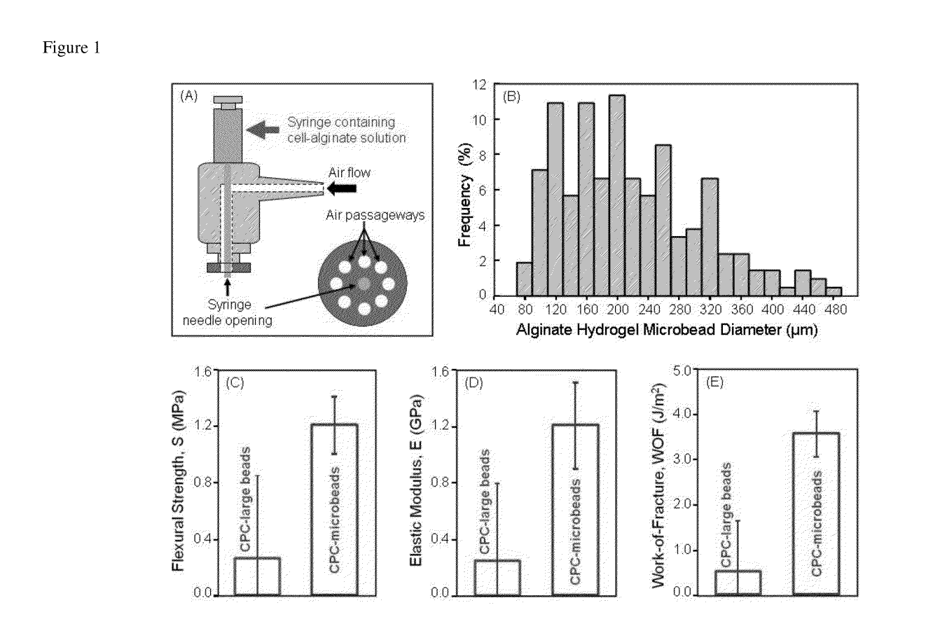

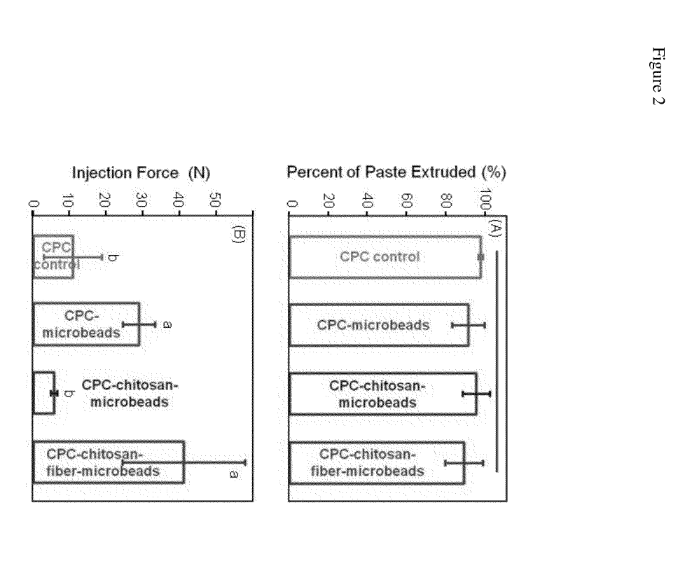

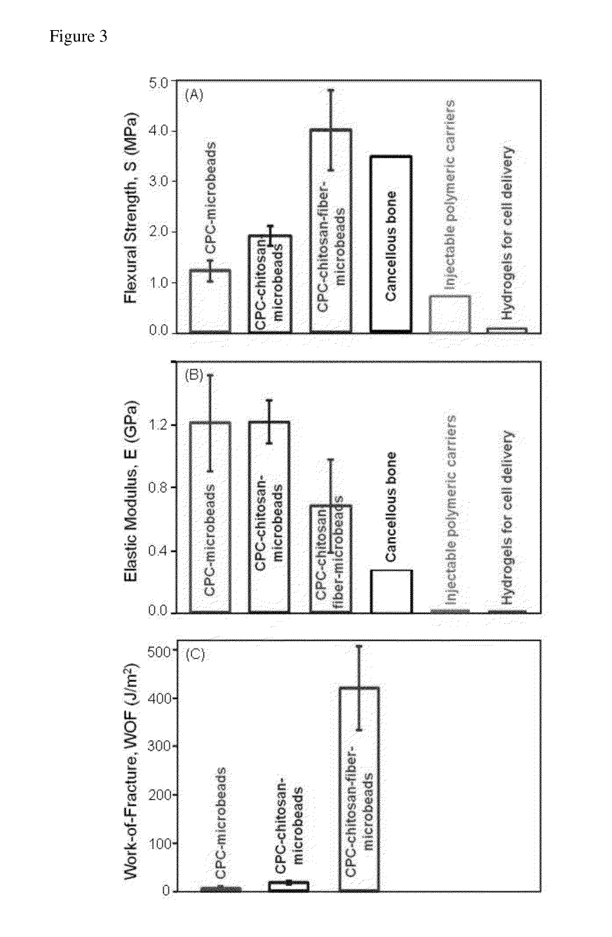

Compared to previous composites with large beads [19,33,34], the CPC-microbead-cell bone pastes described herein have higher mechanical properties (FIG. 1C-E), and are readily injectable (FIG. 2). The addition of fibers increases the injection force. However, the addition of chitosan appears to make the bone paste more cohesive and to lubricate the tetracalcium phosphate (TTCP) and dicalcium phosphate (DCPA) particles of the CPC, thereby decreasing the required injection force. These two factors offset each other, making the injection force of CPC-chitosan-fiber microbead-cell bone pastes statistically similar to that of CPC-microbead-cell bone pastes (FIG. 2B), while the former is much stronger mechanically (FIG. 3). Therefore, the incorporation of chitosan and fibers greatly increases the strength and toughness for the CPC-microbead-cell bone pastes, without compromising the injectability.

A previous study reported a tensile strength of 3.5 MPa for cancellous bone [55]. Other studies have reported a strength of 0.7 MPa for injectable polymeric carriers for cell delivery [56] and 0.1 MPa for hydrogels [57,58]. The elastic modulus for cancellous bone has been reported to be about 0.30 GPa [59]. The reported elastic modulus was 0.008 GPa for an injectable polymeric carrier [56], and 0.0001 GPa for hydrogels [57,58]. These previously described injectable carriers are useful for tissue engineering in non-load-bearing locations only. By contrast, the injectable CPC-chitosan-fiber-microbead-cell bone pastes described herein are much stronger than the previously described injectable carriers (see FIG. 3) and can be used to deliver cells, such as stem cells, in a wide range of craniofacial and orthopedic applications involving bone reconstruction or remodelling. Thus, the strength and modulus of CPC-chitosan-fiber-microbead-cell bone pastes match those of cancellous bone and are much higher than previous injectable polymers and hydrogels for cell delivery. In addition, the hUCMSC-alginate microbead-CPC bone paste is much stronger mechanically than previous composites using large beads.

The hUCMSC-microbead-CPC bone pastes described herein are fully injectable; chitosan and fibers increases the mechanical properties, without compromising the injectability.

The injection process does not interfere with the osteogenic capacity of the cells contained within the bone pastes. For example, encapsulated hUCMSCs differentiate down the osteogenic lineage, as demonstrated by elevated alkaline phosphatase (ALP) and osteocalcin (OC) gene expression, ALP protein synthesis, and mineralization. Expression of osteogenic markers and mineralization of hUCMSCs in the injectable CPC-based bone pastes matches those in hydrogel without CPC.

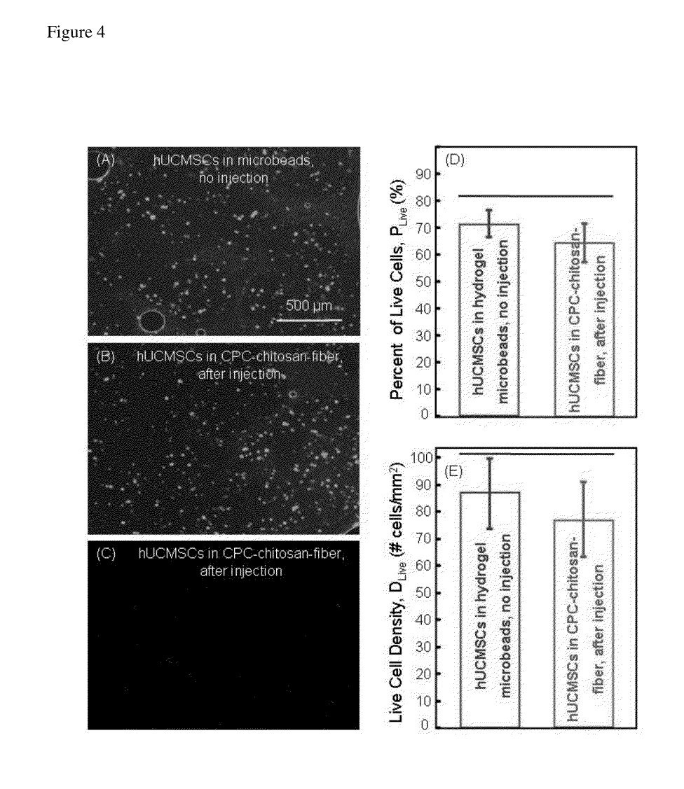

The injection process described herein does not harm the viability of the encapsulated hUCMSCs (FIG. 4). Referring to Examples 1 and 3 herein below, cells inside hydrogel microbeads without CPC, and those in microbeads in CPC, in CPC-chitosan, and in CPC-chitosan-fiber display similar percent of live cells and cell density. (The cells inside alginate hydrogel microbeads without CPC served as a control, because alginate hydrogel is known to be highly biocompatible.) However, hydrogels are weak and not load-bearing. The load-bearing ability was achieved via encapsulation in CPC, without compromising the viability of hUCMSCs. In addition, the FDA-approved CPC (no chitosan, no fibers) served as another biocompatible control. The experiments described herein show that adding chitosan and fibers, which greatly increase the strength and toughness of the bone pastes of the invention, does not adversely affect stem cell viability.

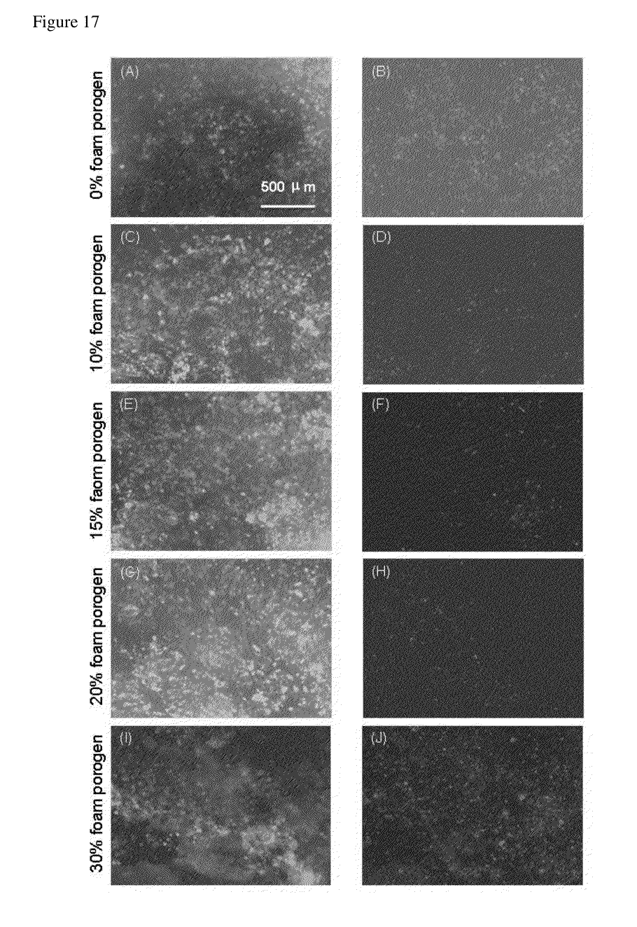

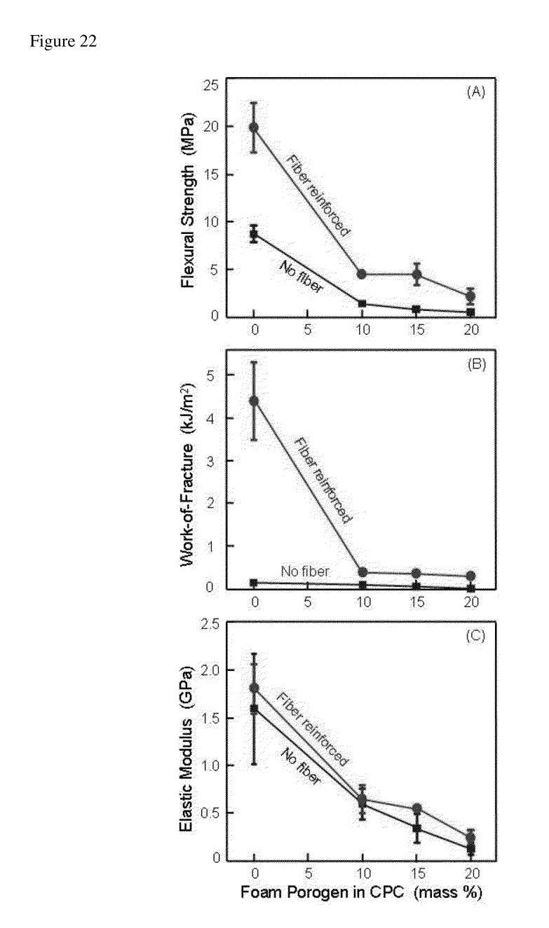

Introducing porosity into the bone pastes of the present invention, such as through the gas-foaming method described in Example 3 below, can serve to improve the viability of cells encompassed with the pastes under particular conditions. CPC that included a porogen showed increased viability, especially when the mass fraction of the porogen in the CPC was between about 15% and 20% (FIG. 17). This is likely due to the higher porosity and interconnectivity providing access of culture media with nutrition and oxygen to the cells inside the CPC. This percentage of live cells is consistent with the reported 70-80% in previous studies on cell encapsulation in hydrogels [64,74]. The strength of a CPC-chitosan-fiber scaffold at 15% gas-foaming porogen was 3.8 MPa (FIG. 22), which approximated the reported 3.5 MPa for cancellous bone [55]. Thus, the CPC used in the bone pastes of the present invention may include porogens.

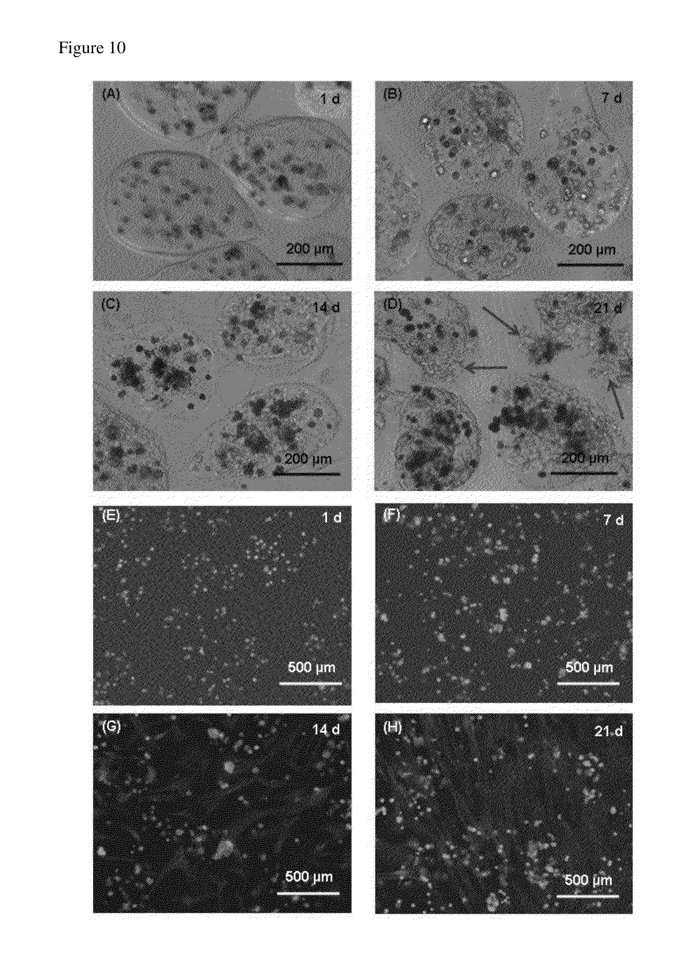

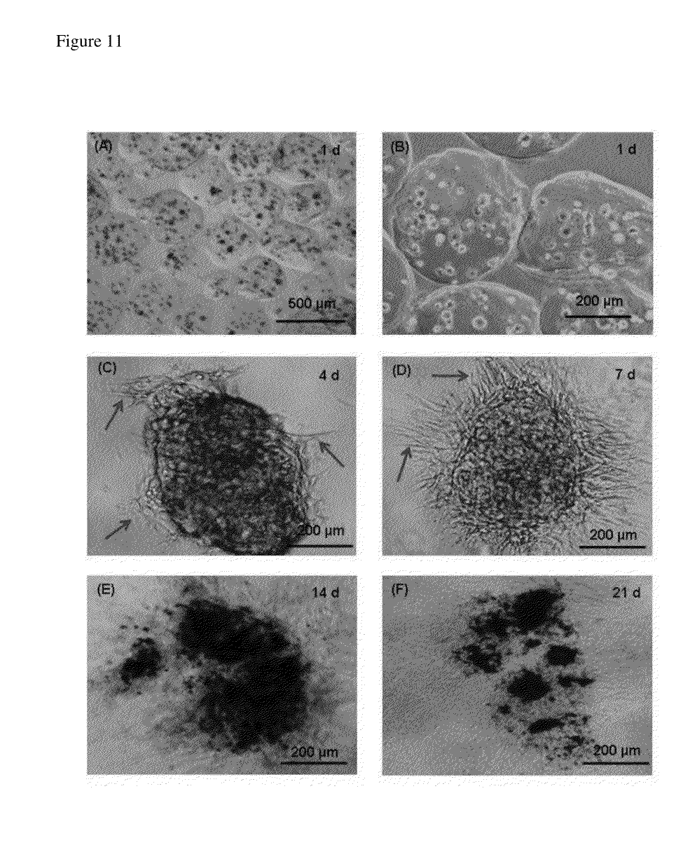

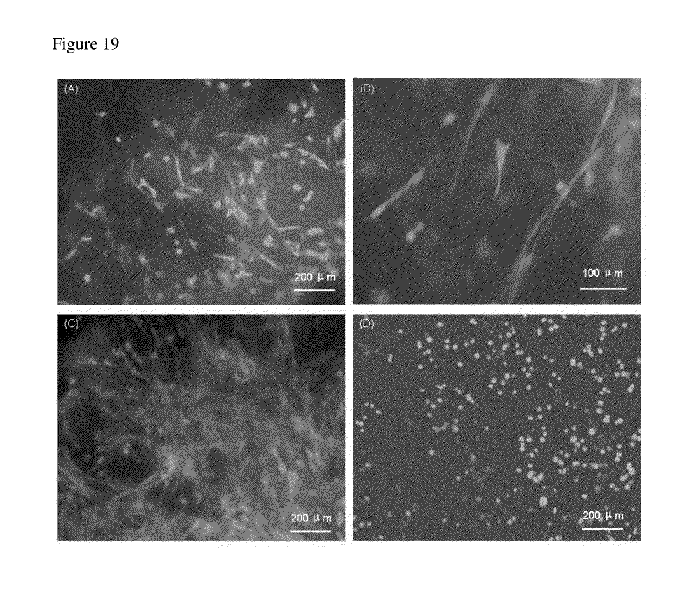

The length of time required for microbead degradation and release of the encapsulated cells can be varied depending on the material used to prepare the beads. In some applications, maintaining the cells within the microbeads for a longer period, such as multiple days, weeks, or even months, can be desirable, while in other applications quick release (within hours or a small number of days) can be preferable. When short-term release of cells is required, alginate-fibrin microbeads may be used. Alginate microbeads comprising a small amount of added fibrin have dramatically increased degradation and decreased the release-of-cell times in culture (FIGS. 10 and 11) and inside the bone paste (FIG. 19). Further, when the cells were released from the degraded microbeads and attached to CPC, they showed a healthy spreading and spindle morphology (FIG. 19A-C), consistent with previous studies on cell attachment on CPC surfaces [70].

In summary:

(1) The new injectable, strong, bone pastes described herein can be used for orthopedic applications and minimally invasive surgeries, with the potential to greatly enhance bone regeneration;

(2) The bone pastes are readily injectable and mechanically strong (in contrast to CPC containing large beads, which is not injectable and is mechanically weak). Indeed, the bone pastes are much stronger mechanically than any currently-available injectable carrier for cell delivery, including polymeric carriers and hydrogels for cell delivery and tissue engineering;

(3) Mechanical properties of the bone pastes can be further strengthened by the inclusion of chitosan and/or fibers, while maintaining the injectability;

(4) The porosity of the bone paste can be controlled through the inclusion of porogenic factors in the CPC;

(5) The timing of microbead degradation and cell release can be varied based on the polymer comprising the microbeads to match specific clinical applications;

(6) The injection process does not harm microbead-encapsulated cells in the bone pastes; and

(7) Stem cells in the microbeads of the bone pastes remain viable, can osteo-differentiate, and can synthesize bone minerals.

Provided below are specific Examples of injectable cell/microbead/CPC bone pastes of the invention. One of ordinary skill in the art will understand that variations of the injectable bone pastes exemplified herein are within the spirit and scope of the invention. For example, as described below, cell types other than hUCMSCs (e.g. but not limited to stem cells such as hBMSCs and hESCs) can be used in the bone pastes of the invention. In addition, the size of the microbeads can be varied. For example, the microbeads used in the Examples below have a mean diameter of approximately 200 μm. By varying the air pressure used, the inventors have produced microbeads of mean diameters from 100 μm to 1500 μm, which are also suitable for injection and creation of macropores in CPC after microbead dissolution. Porogenic factors can be included in the CPC to also controlled the porosity of the bone pastes. The specific Examples described herein employ alginate or alginate-fibrin to fabricate the cell-encapsulating microbeads; however, other hydrogels such as photo-cured hydrogels can be used to make cell-encapsulating microbeads-CPC pastes. The Examples below employ microbeads of 50 vol % in the injectable paste; this volume fraction can be varied as needed. While the CPCs described in the Examples use TTCP and DCPA, the cell-encapsulating microbeads can be readily incorporated into other calcium phosphate cements with various chemistry and compositions. The CPCs described in the Examples employ a TTCP:DCPA molar ratio of 1:1. Other TTCP:DCPA ratios (e.g., but not limited to 1:3), and other fiber types, lengths, and volume fractions can be used [32] in the injectable cell pastes for various orthopedic and other bone repair/remodeling applications. Additional examples of variations that can be employed in the bone pastes described herein are set forth below.

### Calcium Phosphate Cement Compositions

The CPCs used in the bone pastes of the present invention vary based on the identity and proportions of calcium phosphate components that comprise the CPC. Suitable calcium phosphate components include and are not limited to: tetracalcium phosphate (TTCP) (Ca₄(PO₄)₂O), dicalcium phosphate anhydrous (DCPA) (CaHPO₄), dicalcium phosphate dihydrate (CaHPO₄.2H₂O), tricalcium phosphate (Ca₃[PO₄]₂), α-tricalcium phosphate (α-Ca₃(PO₄)₂), β-tricalcium phosphate (β-Ca₃(PO₄)₂), octacalcium phosphate (Ca₈H₂(PO₄)₆. 5H₂O), amorphous calcium phosphate (Ca₃(PO₄)₂), calcium carbonate (CaCO₃), calcium hydroxide (Ca[OH]₂), and hydroxyapatite (Ca₁₀(PO₄)₆(OH)₂), and mixtures thereof. In one aspect of the invention, the CPC comprises TTCP and DCPA. CPCs comprising TTCP and DCPA can contain various molar ratios of TTCP to DCPA. For example, TTCP:DCPA molar ratios can include ratios from about 1:5 to about 5:1, e.g., about 1:5, about 1:4, about 1:3, about 1:2, about 1:1, about 2:1, about 3:1, about 4:1, about 5:1, about 3:2, about 2:3, about 4:3, about 3:4, about 5:4, about 4:5, and others. An example of an acceptable CPC for use in the bone pastes of the present invention comprises a mixture of TTCP:DCPA in a 1:1 molar ratio.

The liquid portion of the CPC can include, for example, water, chitosan, sodium phosphate, hydroxypropyl methylcellulose, and mixtures thereof.

The CPCs used in the bone pastes of the present invention may also comprise a porogen. The inclusion of a porogen permits the formation of macropores in the CPC. A suitable combination that may serve as a porogen in the CPCs of the present invention is sodium hydrogen carbonate (NaHCO₃) and citric acid (e.g., citric acid monohydrate, C₆H₈O₇.H₂O). The acid-base reaction of citric acid with NaHCO₃ produces CO₂ bubbles in CPC, resulting in a macroporous scaffold. NaHCO₃ may be added to the CPC powder at a NaHCO₃/(NaHCO₃+CPC powder) mass fractions of from about 0.5% to about 35%, including from about 5% to about 30%, from about 7.5% to about 25%, and from about 10% to about 20%. NaHCO₃ may also be added to the CPC powder at a NaHCO₃/(NaHCO₃+CPC powder) mass fraction of about: 5%, 7.5%, 10%, 11%, 12%, 13%, 14%, 15%, 16%, 17%, 18%, 19%, 20%, 22.5%, 25%, 27.5%, and 30%. An amount of citric acid sufficient to maintain a fixed NaHCO₃/(NaHCO₃+C₆H₈O₇H₂O) mass fraction of from about 20% to about 70%, including from about 40% to about 60%, and from about 50% to about 60%. Particular examples include a mass fraction of about: 52.5%, 53%, 53.5%, 54%, 54.5%, 54.52%, 55%, 55.5%, 56%, and 56.5%. Additional porogenic factors that may be used include sugar particles, mannitol particles, salt particles and other agents that can dissolve and degrade to create voids in CPC to increase the porosity.

### Chitosan

The CPCs of the present invention may also contain chitosan. Chitosan content can vary from about 0% to about 50% by mass, for example (but not limited to), 0% to about 5%; about 5 to about 10%; about 10% to about 15%; about 15 to about 20%; about 20 to about 25%; about 25 to about 30%; about 30 to about 35%; about 35 to about 40%; about 40 to about 45%; about 45 to about 50%, by mass, e.g., 1%, 2%, 3%, 4%, 5%, 6%, 7%, 8%, 9%, 10%, 11%, 12%, 13%, 14%, 15%, 16%, 17%, 18%, 19%, 20%, 25%, 30%, 35%, 40%, 45%, 50%.

### Fibers

The CPCs of the present invention may also contain fibers to strengthen and/or reinforce the bone pastes. Fibers that can be used in the bone pastes of the invention include and are not limited to: rods, fibers, ropes, threads, or meshes. The fibers can be, e.g., glass fibers, ceramic fibers, polymer fibers, metal fibers, or mixtures thereof. Examples include and are not limited to: poly(L-lactide)-based polymer fibers, glycolic acid-based polymers, and poly(D,L-lactic acid) fibers. The fiber diameters can range from about 100 nm to about 1 mm. The length can range from about 0.1 mm to about 10 mm. The fibers can be non-degradable or degradable. The volume fraction (vol %) of fibers used can be, e.g., about 0.5% to about 50%, e.g., about: 0.5-5, 5-10, 10-15, 15-20, 20-25, 25-30, 30-35, 35-40, 40-45, and 45-50 vol %, and about: 5 vol %, 10 vol %, 12.5 vol %, 15 vol %, 17.5 vol %, 20 vol %, 22.5 vol %, 25 vol %, 27.5 vol %, 30 vol %, 32.5 vol %, 35 vol %, 37.5 vol %, 40 vol %, 42.5 vol %, 45%, 47.5 vol %, and 50 vol %. The concentration of fibers can differ based on the diameter of the fibers as large-diameter fibers that are relatively stiff are less injectable, finer fibers are more flexible and may be more injectable. Vol % equals the volume of fibers/the volume of the complete bone paste. An example of a suitable fiber is an absorbable suture fiber, such as Vicryl™, from Ethicon, Somerville, N.J.

### Bioactive Agents

The CPCs of the present invention may also contain one or more bioactive agents. There is no particular limitation on the identity of agents that may be utilized, but examples include agents that induce migration of cells to the locus of bone paste application, agents that induce maturation and/or differentiation of cells in the locus of bone paste application, and agents that promote the growth, differentiation, attachment and/or proliferation of the cells encapsulated within the bone paste. Suitable agents include, but are not limited to, cytokines, growth factors, bone morphogenic proteins (e.g. BMP-1, BMP-2, BMP-3, BMP-4, BMP-5, BMP-6, BMP-7, and BMP-8a), hormones, steroids, extracellular matrix components, anesthetics, analgesics, opioids, anti-inflammatory agents, including anti-inflammatory steroid or non-steroidal anti-inflammatory agents, enzyme inhibitors, immunosuppressive agents, growth hormone antagonists, radio- and chemo-therapeutic agents, antimicrobial agents, antibiotics, anti-parasite and/or anti-protozoal compounds, muscle relaxants, anti-spasmodics and muscle contractants including channel blockers, miotics and anti-cholinergics, actin inhibitors, remodeling inhibitors, cell growth inhibitors, anti-adhesion molecules, vasodilating agents, anti-pyretics, anti-angiogenic factors, anti-secretory factors, anti-coagulants and/or anti-thrombotic agents, inhibitors of DNA, RNA or protein synthesis, peptides, proteins, enzymes, lubricants, and imaging agents. In a certain embodiments, the bioactive agent is a drug.

### Polymers for Cell Encapsulation

The bone pastes of the present invention contain microbeads that encapsulate stem cells. Hydrogels and biocompatible polymers for cell encapsulation include and are not limited to: alginate, partially oxidized alginate, oxidized alginate, alginate-fibrin, partially oxidized alginate-fibrin, oxidized alginate-fibrin, poly(ethylene glycol diacrylate), poly(ethylene glycol)-anhydride dimethacrylate, gelatin, chemically cross-linked polymers, ionically cross-linked polymers, heat-polymerized polymers, and photopolymerized polymers.

In one aspect, oxidized alginate-fibrin microbeads are used, wherein fibrinogen is added to an alginate solution at a fibrinogen mass fraction of from about 0.05% to about 1% to render microbeads that have sufficient mechanical integrity and that are readably degradable. The fibrinogen may also be added to the alginate solution at a fibrinogen mass fraction of about: 0.05%, 0.06%, 0.07%, 0.08%, 0.09%, 0.10%, 0.11%, 0.12%, 0.13%, 0.14%, 0.15%, 0.16%, 0.18%, 0.20%, 0.22%, 0.24%, 0.26%, 0.28%, 0.30%, 0.32%, 0.34%, 0.36%, 0.38%, 0.40%, 0.42%, 0.44%, 0.46%, 0.48%, 0.50%, 0.52%, 0.54%, 0.56%, 0.58%, 0.60%, 0.62%, 0.64%, 0.66%, 0.68%, 0.70%, 0.72%, 0.74%, 0.76%, 0.78%, 0.80%, 0.82%, 0.84%, 0.86%, 0.88%, 0.90%, 0.92%, 0.94%, 0.96%, 0.98%, or 1%.

The alginate-fibrin microbeads may be produced by first preparing an oxidized alginate solution by dissolving sodium alginate in water, and adding sodium periodate to induce an oxidization reaction. Ethanol-precipitated product is dissolved in saline, and fibrinogen is added to the solution to yield a mixed alginate-fibrinogen solution. Cells are then added to the oxidized alginate-fibrinogen solution, such as at a density of 1×10⁶ cells/mL. The alginate-fibrinogen droplets are produced as described in the Examples and sprayed into a solution of a calcium chloride and thrombin, where the calcium chloride induces alginate cross-linking, while a reaction between fibrinogen and thrombin produces fibrin.

Hydrogels and biocompatible polymers for cell encapsulation can be formed into microbeads of various diameters for use in the bone pastes described herein. For example, average microbead diameters including but not limited to about 50 μm to about 1500 μm can be used, e.g., about: 50 μm, 75 μm, 100 μm, 200 μm, 300 μm, 400 μm, 500 μm, 600 μm, 700 μm, 800 μm, 900 μm, 1000 μm, 1100 μm, 1200 μm, 1300 μm, 1400 μm, 1500 μm, and various ranges and mixtures thereof. Smaller and larger bead sizes may be used, as long as they the beads can be easily injected without compromising cell viability. In general, beads should be less than 2 millimeters, so as to minimize cell damage caused by injection of the bone paste.

Examples of volume fractions (vol %) of microbeads for use in the bone pastes described herein are (and are not limited to): about 10% to about 80%, e.g., about: 40-45%, 45-50%, 50-52%; 50-55%, 55-60%, 40-50%, 50-60%, 55-65%, 60-70%, 65-75%, 70-80%, and about: 40%, 45%, 50%, 55%, 60%, 65%, 70%, 75%, 80%, and the like. Vol % equals the volume of microbeads/the volume of the complete bone paste.

### Cells

Cells that can be encapsulated in the bone pastes of the invention include and are not limited to: bone-growing cells, blood vessel-growing cells, and cartilage-growing cells, such as mesenchymal stem cells, embryonic stem cells, umbilical cord stem cells, bone marrow stem cells, lymphoid stem cells, myeloid stem cells, stromal cells, osteogenic cells, osteoblast cells, chondrogenic cells, angiogenic cells, endothelial cells, and mixtures thereof. The noted stem cells include totipotent stem cells, pluripotent stem cells, induced pluripotent stem cells, multipotent stem cells, oligopotent stem cells, unipotent cells, progenitor cells, and osteoblasts. Cells can be human or from any other suitable animal.

Various cell densities can be used in the construction of the microbeads encapsulating the cells, e.g. but not limited to, about 10⁴ cells/ml of polymer solution to about 5×10⁶ cells/ml of polymer solution, e.g., about: 10⁴, 10⁵, 5×10⁵, 10⁶, 2.5×10⁶, 5×10⁶, etc. E.g., one useful range of cell densities is about 5×10⁵ to about 2×10⁶, e.g., about 10⁶. After injection of a bone paste containing the cells, cell viability is preferably at least about 50%, e.g, at least about: 50%, 55%, 60%, 65%, 70%, 75%, 80%, 85%, and the like, compared to cell viability before injection.

### Bone Pastes Kits

The bone pastes of the present invention may be used in a variety of applications and settings. It is clear that one setting is a physician's office or an operating room where the paste is applied to a site of bone injury. The bone pastes may be provided in kits for use in such circumstances where components of the bone pastes are included in the kit and combined on site. The components of such kits may include:

1) A powder and a liquid

2) Cell-encapsulating microbeads (either from a frozen stock, or freshly encapsulated)

3) Mixing pad, spatula, and an injection syringe system

It will be clear that kits comprising variations on the listed components are encompassed within the scope of the invention and that such variations will be readily apparent to the skilled artisan.

A specific embodiment of the present invention is more particularly described in the following Example, which is intended as illustrative only, since numerous modifications and variations thereof will be appreciated by those of ordinary skill in the art.

### EXAMPLES

### Example 1

### 1A. Materials and Methods

1.1. Encapsulation of hUCMSCs in Alginate Hydrogel Beads

While set CPC has excellent biocompatibility, the paste mixing and setting reaction harm the cells. Therefore, alginate was used to encapsulate and protect the cells, selected because alginate is biocompatible and can form a crosslinked gel under mild conditions [36]. A 1.2% (mass fraction) sodium alginate solution was prepared by dissolving alginate (MW=75,000 to 220,000 g/mol, ProNova, Norway) in saline (155 mmol/L NaCl) [33,34]. Alginate is a natural polysaccharide extracted from seaweed, and is made up of blocks of guluronic acid and mannuronic acid along the polymer backbone. This alginate is ultrapure with a low viscosity, and with 64% guluronic acid and 36% mannuronic acid blocks. When alginate gels, it is the guluronic acid blocks that are crosslinked together via the calcium ions from calcium chloride.

hUCMSCs were generously provided by Dr. M. S. Detamore (University of Kansas, Lawrence, Kans.). hUMSCs can also be obtained commercially (for example, from ScienCell Research Laboratories, human umbilical cord mesenchymal stem cells #7530, Carlsbad, Calif.).

hUCMSCs were harvested from the Wharton's jelly of umbilical cords as described previously [11,16]. Briefly, umbilical cords were obtained from an obstetrician and incubated in a collagenase type I solution containing collagenase type I (300 U/mL) (Sigma, St. Louis, Mo.), hyaluronidase (1 mg/mL) (MP Biomedical, Aurora, Ohio), and calcium chloride (3 mM) (Fisher, Pittsburgh, Pa.), for 30 min at 37° C. Then, the vascular tissue was removed, and the cords were minced and plated in a modified Dulbecco's modified Eagle's medium (DMEM) for 1 week [14]. The cord remnants were then removed and the attached cells were harvested.

Cells were cultured in a low-glucose DMEM with 10% fetal bovine serum (FBS) and 1% penicillin/streptomycin (PS) (Invitrogen, Carlsbad, Calif.) (control media) [17]. At 80-90% confluence, hUCMSCs were detached by trypsin and passaged. Passage 4 hUCMSCs were used for experiments. The osteogenic media had 100 nM dexamethasone, 10 mM β-glycerophosphate, 0.05 mM ascorbic acid, and 10 nM 1α,25-Dihydroxyvitamin (Sigma) [16,19].

To fabricate hydrogel beads containing the hUCMSCs, cells were encapsulated in alginate at a density of 1 million cells/mL of alginate solution [19]. Bead formation was accomplished by extruding alginate-cell droplets through a sterile syringe into a well of 100 mmol/L calcium chloride solution. The alginate droplets crosslinked and formed beads. This resulted in beads of 2-3 mm in diameter, which are the same as those in previous studies [19,33,34], and are referred to as “large beads”.

To improve the injectability of CPC-beads paste, the inventors developed small beads encapsulating hUCMSCs. The alginate-cell solution was loaded into a sterile syringe which was placed into a syringe pump and connected to a bead generation device (Var J1, Nisco, Zurich, Switzerland), as shown schematically in FIG. 1A. Nitrogen gas was fed to the gas inlet and a pressure of 10 psi was established to form a coaxial air flow to break up the alginate droplets. This produced small alginate beads that crosslinked in the calcium chloride bath. A microscope (Eclipse TE-2000S, Nikon, Melville, N.Y.) with a CCD camera (DS-Qil, Nikon) was used to measure the sizes of the beads, which averaged 207 μm. These small beads are designated “microbeads”.

1.2. CPC, Chitosan, and Degradable Fibers

CPC consisted of tetracalcium phosphate (TTCP; Ca₄(PO₄)₂O) and dicalcium phosphate anhydrous (DCPA; CaHPO₄) [31,32]. TTCP was synthesized from a solid-state reaction between DCPA and CaCO₃, then ground in a blender to obtain particle sizes of 1-80 μm (median=17 μm). DCPA was ground to obtain particle sizes of 0.4-3.0 μm (median=1.0 μm). The TTCP and DCPA powders were mixed at a molar ratio of 1:1 to form the CPC powder. The purpose of adding chitosan to CPC was to render it fast-setting and strong [37]. Chitosan and its derivatives are natural biopolymers found in arthropod exoskeletons; they are biodegradable and osteoconductive [38]. Chitosan lactate (Vanson, Redmond, Wash.) was mixed with water at a chitosan/(chitosan+water) mass fraction of 15% to form the CPC liquid, referred to as “chitosan liquid” [39]. An absorbable suture fiber (Vicryl, polyglactin 910, Ethicon, Somerville, N.J.) was used because it possessed a relatively high strength [40]. Individual fibers about 14 μm in diameter were braided to form the suture with a bundle diameter of about 300 μm [40]. This suture fiber reinforced CPC for about four weeks, and then dissolved and created long macropores in CPC. The fiber was cut to a length of 3 mm so that the CPC-fiber paste was injectable, based on a preliminary experiment.

1.3. Injectability

A 10 mL syringe (Free-Flo, Kerr, Romulus, Mich.) with an internal diameter of 10 mm was used with a 10-gauge needle having an inner diameter of 2.7 mm [41,42]. The 10-gauge needle was similar to spinal needles used in the augmentation of osteoporotic vertebrae and the management of vertebral compression fractures [43]. A CPC composite paste of 3 g (with compositions described below) was used for each injection test [41]. The paste was mixed with a spatula for 30 s and then filled into the syringe. The syringe was placed between the compression plates of a computer-controlled Universal Testing Machine (MTS, Eden Prairie, Minn.). Following a previous study [42], 1.5 min after the start of mixing, the compression was started and the cement was extruded at a crosshead speed of 15 mm/min. This was continued until either the entire paste was extruded, or a maximum force of 100 N was reached at which point the test was stopped [42]. A preliminary study showed that it took at least 5 min for the paste to undergo significant setting. In the injection, it took 1 min or less to finish injecting each paste, hence setting in the syringe was minimal. The percentage of paste extruded was determined as the mass of the extruded paste/the original mass of the paste inside the syringe [41]. The injection force was continuously recorded, and the maximum force was referred to as the injection force [41].

The following materials were tested for injectability, all at the same CPC powder to liquid ratio of 2/1 by mass: (1) CPC with water (control); (2) CPC with water+50% volume fraction (vol %) of hydrogel microbeads (designated as CPC-microbeads); (3) CPC with chitosan+50 vol % of microbeads (CPC-chitosan-microbeads); (4) CPC with chitosan+50 vol % of microbeads+20 vol % fibers (CPC-chitosan-fiber-microbeads).

For the microbeads, the 50 vol % was equal to the volume of microbeads/the volume of the entire specimen. This could encapsulate a relatively large amount of cells, and create 50 vol % of macroporosity in CPC after the dissolution of the beads. For the fibers, the 20 vol % was equal to the volume of fibers/the volume of the entire specimen. This volume of fibers was selected because preliminary study showed that CPC with 10-20 vol % of fibers were readily injectable, whereas CPC with 25 vol % fibers was difficult to inject. CPC with large beads, the same as those in previous studies [19,33,34], was not included here because it was difficult to mix the large beads homogeneously with the paste, the paste was difficult to inject, and the large beads broke when forced through the 10-gauge needle.

1.4. Mechanical Testing

The CPC composite paste with cell-encapsulating hydrogel beads was filled to a mold of 3×4×25 mm. Each specimen was set in a humidor for 4 h at 37° C. The hardened specimen was demolded and immersed in the culture media for 1d. A three-point flexural test was used to fracture the specimens on the Universal Testing Machine [44]. Flexural strength S=3FₘₐₓL/(2bh²), where Fₘₐₓ is the maximum load on the load-displacement (F-d) curve, L is span, b is specimen width, and h is thickness. Elastic modulus E=(F/d) (L³/[4bh³]), where load F divided by displacement d is the slope. Work-of-fracture (toughness), WOF, was calculated as the area under the F-d curve divided by the specimen's cross-sectional area [40].

1.5. Viability of Encapsulated hUCMSCs

hUCMSCs were encapsulated in microbeads as described in Section 1 above. For viability, first, comparison was made between: (1) cells in microbeads without injection, and (2) cells in microbeads in CPC-chitosan-fiber paste after injection. Both were cultured for 1d. The purpose was to determine whether the paste mixing and injection process would harm the cells. Next, the effects of different compositions of the injectable construct were compared as follows: (1) hUCMSCs in hydrogel microbeads; (2) hUCMSCs in microbeads in CPC; (3) hUCMSCs in microbeads in CPC-chitosan; (4) hUCMSCs in microbeads in CPC-chitosan-fiber paste. hUCMSCs in hydrogel microbeads (without CPC) served as a control, because alginate hydrogel is known to have excellent biocompatibility. However, the microbeads are not load-bearing. Therefore, the purpose of using CPC-based scaffolds was to deliver hUCMSCs in injectable and load-bearing constructs, without compromising the cell function. Each construct was set in a cell culture well at 37° C. for 30 min. Then, 2 mL of the osteogenic media was added to each well. At 1, 7, and 14 d, the constructs were carefully broken and the cell-encapsulating microbeads were harvested. Cells were live/dead stained with a kit (Invitrogen, Carlsbad, Calif.). The percentage of live cells was: P_{Live}=N_{Live}/(N_{Live}+N_{Dead}), where N_{Live}=the number of live cells, and N_{Dead}=the number of dead cells [45]. In addition, the live cell density, D_{Live}, was calculated: D_{Live}=N_{Live}/A, where A is the area of the view field for N_{Live [}17,19].

1.6. Osteogenic Differentiation of Encapsulated hUCMSCs

For osteodifferentiation, three experiments were performed on four constructs: (1) hUCMSCs in microbeads; (2) hUCMSCs in microbeads in CPC; (3) hUCMSCs in microbeads in CPC-chitosan scaffold; (4) hUCMSCs in microbeads in CPC-chitosan-fiber scaffold. Experiment I measured the osteogenic gene expression of hUCMSCs using quantitative real-time reverse transcription polymerase chain reaction (qRT-PCR, 7900HT, Applied Biosystems, Foster City, Calif.). The encapsulated hUCMSCs were cultured in the constructs for 1, 4, 7, and 14 days. The total cellular RNA of the encapsulated hUCMSCs were extracted with TRIzol reagent (Invitrogen) and reverse-transcribed into cDNA using a High-Capacity cDNA Archive kit. TaqMan gene expression assay kits, including two pre-designed specific primers and probes, were used to measure mRNA transcript levels for human alkaline phosphatase (ALP, Hs00758162_m1), Osteocalcin (OC, Hs00609452_g1), and glyceraldehyde 3-phosphate dehydrogenase (GAPDH, Hs99999905). Relative expression level for each target gene was evaluated using the 2-ΔΔCt method [46]. The Ct values of target genes were normalized by the Ct values of the TaqMan human housekeeping gene GAPDH to obtain the ΔCt values. These values were then subtracted by the Ct value of the hUCMSCs cultured on tissue culture polystyrene in the control media for 1 day (the calibrator) to obtain the ΔΔCt values. The fold of change was obtained with n=5.

Regarding culture time, in general, cells were cultured for 1, 7, and 14 d, following previous studies [34,39,45]. An exception was made for RT-PCR, where day-4 was added. This is because osteogenesis-associated gene expression occurs in early osteodifferentiation, and a previous study observed ALP expression peaking at 4 d [46]. Another exception was made for ALP production by the cells, where day-1 was not done but day-21 was added. This is because the ALP enzyme production by the cells occurs at a later time; it peaked at 14-d in previous studies [19,45]. Doing 7, 14 and 21d would help determine if the ALP synthesis peaks at 14 d or 21d.

Experiment II measured the hUCMSCs' synthesis of the ALP enzyme. The cell-encapsulating microbeads harvested after 7, 14, and 21d were dissolved by 55 mmol/L sodium citrate tribasic solution (Sigma). A colorimetric p-nitrophenyl phosphate (pNPP) assay (Stanbio, Boerne Tex.) was used to measure the ALP activity. Normal control serum containing a known concentration of ALP was used as a standard. A microplate reader (M5 SpectraMax, Molecular Devices, Sunnyvale, Calif.) was used and the ALP was normalized by the DNA content [19,45]. DNA was quantified using the Quant-iT PicoGreen Kit (Invitrogen, Carlsbad, Calif.) following standard protocols [19,45].

Experiment III examined the mineral synthesis by the encapsulated hUCMSCs. Minerals emit red fluorescence when stained with xylenol orange (Sigma). The minerals synthesized by the cells in the hydrogel microbeads harvested from the constructs were stained and examined using both phase contrast and fluorescence images. Following a previous study [19], the mineral area percentage was calculated as A_{Mineral}/A_{Total}, where A_{Mineral} is the area of mineralization (red fluorescence), and A_{Total} is the total area of the field of view of the image.

One-way and two-way ANOVA were performed to detect significant effects of the variables. Tukey's multiple comparison tests were used at a p value of 0.05.

### 1B. Results

FIG. 1B shows the diameter histogram of the hUCMSC-encapsulating hydrogel microbeads, based on the measurement of 211 random beads. The diameter ranged from 73 to 465 μm, with (mean±sd) of (207±89)

The mechanical properties of CPC constructs containing 50 vol % of hUCMSC-encapsulating hydrogel beads are plotted in FIG. 1C-E. Compared to the large beads similar to those in previous studies, the use of microbeads increased the flexural strength of the CPC construct by 4-fold, elastic modulus by 5-fold, and work-of-fracture by 7-fold.

The injectability results are shown in FIG. 2. All the pastes were readily extruded at relatively small injection forces. Compared to CPC control (no chitosan, no beads, no fibers), CPC+50% microbeads was extruded at a higher injection force (p<0.05). Adding chitosan made the paste more cohesive, which was fully extruded at a substantially lower force (p<0.05). The paste with 20% fibers was still injectable, but the injection force was higher than that for CPC-chitosan-microbeads without fibers (p<0.05). Compared to CPC-microbeads, the injection force for CPC-chitosan-fiber-microbeads was similar (p>0.1); therefore, adding chitosan and fibers for reinforcement did not compromise the injectability.

The mechanical properties of CPC constructs containing 50% of hUCMSC-encapsulating microbeads are plotted in FIG. 3. Adding chitosan and fibers increased the load-bearing properties (p<0.05). CPC-chitosan-fiber-microbeads reached a flexural strength of about 4 MPa. Compared to CPC-microbeads, the flexural strength of CPC-chitosan-fiber-microbeads was 3-fold higher, and the work-of-fracture was 100-fold higher. The elastic modulus decreased because the polyglactin fibers were flexible and not stiff. The previously-reported strength and modulus values for cancellous bone, injectable polymer and hydrogels for cell delivery are included in FIG. 3, as described in herein.

FIG. 4 compares the hUCMSCs in hydrogel microbeads alone (without CPC and without injection), and those mixed with the CPC-chitosan-fiber paste and then injected. Both were cultured for 1d and then stained. Live cells were stained green and were numerous (A and B). Dead cells were stained red and were few (C). In (D) and (E), the percentage of live cells and cell density values indicate that the paste mixing and injection process did not significantly harm the encapsulated hUCMSCs.

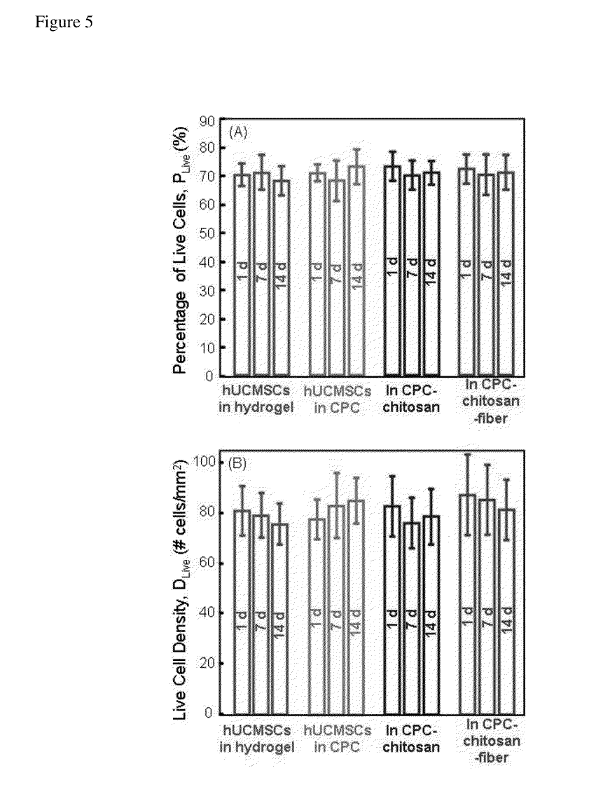

Next, different construct compositions were compared, and the culture was prolonged to 7 d and 14 d. The percentage of live cells and cell density are plotted in FIG. 5. In each plot, all the values were statistically similar (p>0.1). Therefore, compared to hUCMSCs in hydrogel microbeads without CPC, encapsulation in CPC did not compromise the cell viability. In addition, adding chitosan and fibers for reinforcement did not adversely affect the cell viability compared to the CPC without chitosan or fibers. Furthermore, the hUCMSCs remained viable during encapsulation at 14 d.

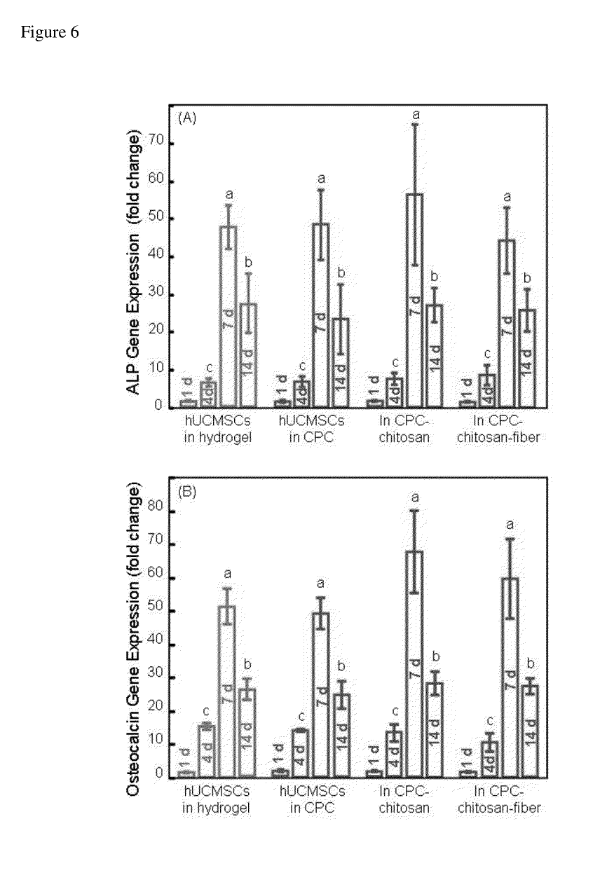

Three experiments were performed to investigate the osteogenic differentiation of the encapsulated hUCMSCs. The results of experiment I is plotted in FIG. 6. The ALP gene expression was significantly increased at 4 d over that at 1d, greatly increased at 7 d, and then slightly decreased at 14 d. The OC showed a similar trend. Compared to hUCMSCs in hydrogel microbeads without CPC, encapsulation in CPC did not adversely affect the ALP or OC expression levels (p>0.1). In addition, adding chitosan and fibers for reinforcement did not compromise the ALP or OC expression levels, compared to those of CPC without chitosan and fibers (p>0.1).

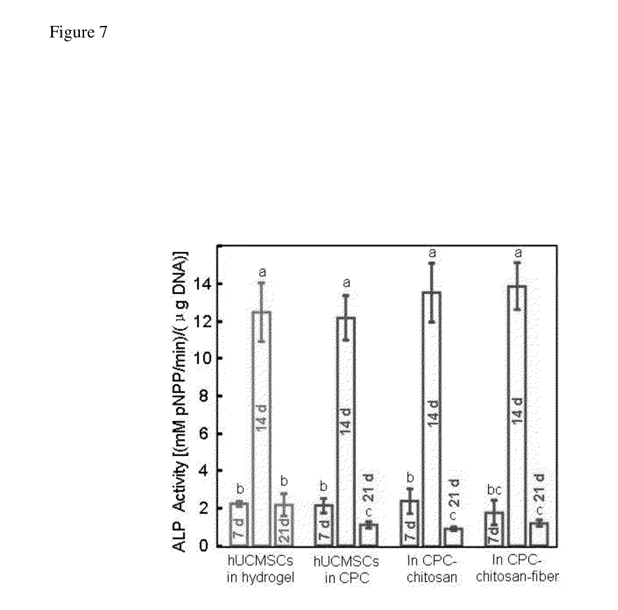

The results of Experiment II are plotted in FIG. 7. The ALP enzyme production by the hUCMSCs greatly increased at 14 d over that at 7 d, and then decreased at 21d (p<0.01). The ALP at 14 d was about 6-fold higher than that at 7 d for all the constructs.

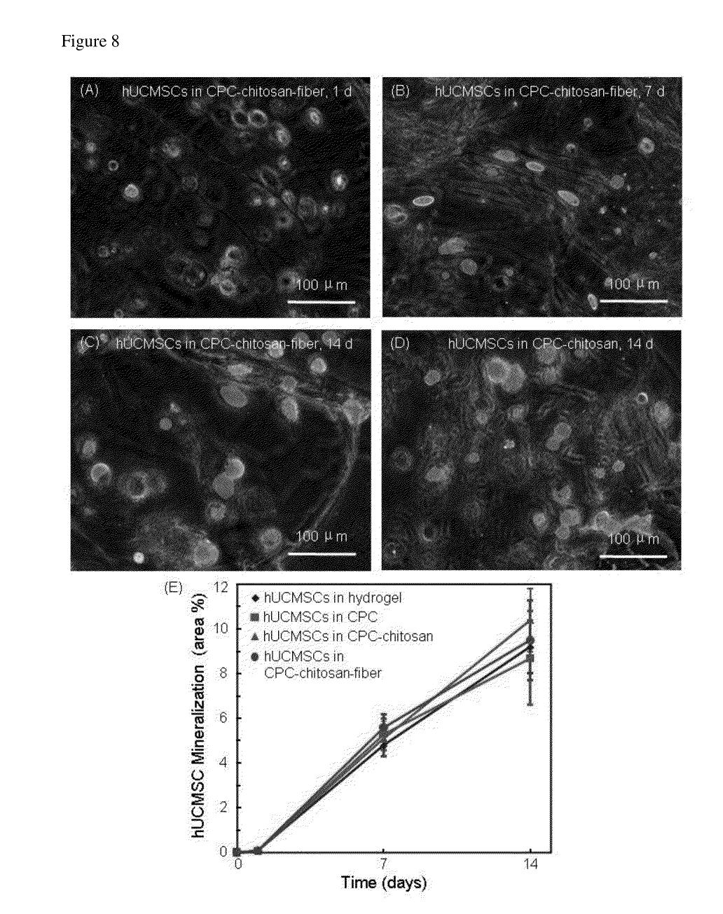

The results of Experiment III are shown in FIG. 8. In (A-C), the staining of minerals synthesized by hUCMSCs in CPC-chitosan-fiber construct increased progressively from 1d, to 7 d, and to 14 d. This trend was the same in all four constructs. An example for CPC-chitosan construct is shown in (D). The stained mineral area percentage is plotted in (E). The staining areas at 7 d were 50-fold higher than those at 1d. The staining areas at 14 d were nearly 2-fold higher than those at 7 d. Encapsulation in CPC did not lower the mineral synthesis of hUCMSCs compared to that in hydrogel microbeads alone (p>0.1). The incorporation of chitosan and fibers, which greatly increased the load-bearing capability of the injectable construct, did not decrease the hUCMSCs' mineral synthesis capability (p>0.1).

### Example 2

### 2A. Materials and Methods

2.1. hUCMSC Culture

hUCMSCs were obtained commercially (ScienCell, Carlsbad, Calif.). They were harvested from the Wharton's Jelly in human umbilical cords of healthy babies, using an established method [11,14]. Cells were cultured in a low-glucose Dulbecco's modified Eagle's medium (DMEM), supplemented with 10% fetal bovine serum (FBS) and 1% penicillin-streptomycin (Invitrogen, Carlsbad, Calif.), and this media is termed “control media”. At 80-90% confluence, hUCMSCs were detached and passaged, and passage 4 cells were used. The osteogenic media consisted of the control media supplemented with 100 nM dexamethasone, 10 mM β-glycerophosphate, 0.05 mM ascorbic acid, and 10 nM 1α,25-Dihydroxyvitamin (Sigma) ([13, 16, 19].

2.2. Synthesis of Hydrogel Microbeads with hUCMSC Encapsulation

Hydrogels are appealing as carriers for cell encapsulation and delivery because of their structural similarity to the extracellular matrix of tissues and their high water and fluid content for cell survival. Alginate has been used to encapsulate cells because it is non-cytotoxic and can form an ionically-crosslinked network under mild conditions [33, 34, 19]. Previous studies encapsulated stem cells in alginate microbeads with good viability [19,68]. In the present study, three types of alginate-based microbeads were fabricated to encapsulate the hUCMSCs: (1) Alginate microbeads; (2) oxidized alginate microbeads; (3) oxidized alginate-fibrin microbeads.

For type 1, a 1.2% (mass fraction) sodium alginate solution was prepared by dissolving alginate (UP LVG, 64% guluronic acid, MW=75,000 to 220,000 g/mol, ProNova Biomedical, Oslo, Norway) in saline (155 mmol/L NaCl), following previous studies [34,71]. hUCMSCs were added to the alginate solution at a density of 1 million cells/mL of alginate. The alginate-cell solution was loaded into a syringe, which was placed into a pump and connected to a bead-generating device (Var J1, Nisco, Zurich, Switzerland). Nitrogen gas was fed to the inlet and a pressure of 10 psi was established to form a coaxial air flow to break up the alginate droplets to obtain fine microbeads. The droplets fell into a well of 100 mmol/L calcium chloride solution and crosslinked to form microbeads [19].

For type 2, the alginate was partially oxidized to increase its degradability. The oxidation reaction was done using sodium periodate at the correct stoichiometric ratio of sodium periodate to alginate to yield a certain percentage of alginate oxidation [63]. The percentage of oxidation (%) was the number of oxidized uronate residues per 100 uronate units in the alginate chain. In a previous study, alginate of up to 5% oxidation was synthesized [63]. In our preliminary studies, the microbeads using 5% oxidized alginate were undegraded and did not release the cells after 21 d in the culture media. The microbeads using 10% oxidized alginate were too weak to be handled and could easily be damaged, and hence could not protect the cells. Hence, in the present study, alginate at 7.5% oxidation was synthesized. The alginate oxidation followed previous procedures [63]. Briefly, 1% by mass of sodium alginate was dissolved in distilled water. 1.51 mL of 0.25 M sodium periodate (Sigma, St. Louis, Mo.) was added to 100 mL of alginate solution, which was stirred to react in the dark at room temperature. At 24 h, the oxidization reaction was stopped by adding 1 g of ethylene glycol and then 2.5 g of sodium chloride. Ethanol of 200 mL was added to precipitate the product, which was then collected by centrifugation. The precipitates were re-dissolved in 100 mL of water and precipitated with 200 mL of ethanol. The second precipitates were collected and dissolved in 30 mL of water. The final product was freeze dried for 24 h, and used to make the microbeads. The oxidized alginate thus obtained was dissolved in saline at a concentration of 1.2% by mass, and the hUCMSC-encapsulating microbeads were made as described for type 1. They are referred to as “oxidized alginate microbeads”.

For type 3, fibrin was added to the oxidized alginate to obtain oxidized alginate-fibrin microbeads. An oxidized alginate solution at a concentration of 1.2% by mass in saline was prepared as described for type 2. Fibrinogen from bovine plasma (Sigma) was added at a concentration of 0.1% to the alginate solution and incubated at 37° C. for 2 h to yield a mixed alginate-fibrinogen solution. The fibrinogen concentration of 0.1% was selected because in preliminary studies, fibrinogen >0.1% yielded microbeads that were sticking to each other because fibrin was sticky. Fibrinogen concentration <0.1% resulted in microbeads that were not fast degradable. hUCMSCs were added to the alginate-fibrinogen solution at a density of 1×10⁶ cells/mL. For cross-linking, a solution containing 150 mL of 100 mmol/L calcium chloride plus 125 NIH units of thrombin (Sigma) was prepared. When the alginate-fibrinogen droplets were sprayed into this solution, calcium chloride caused the alginate to crosslink, while the reaction between fibrinogen and thrombin produced fibrin. This yielded hUCMSC-encapsulating microbeads that are referred to as “oxidized alginate-fibrin microbeads”.

2.3. Viability of hUCMSCs

For each type of hUCMSC-encapsulating microbeads, 1 mL of microbeads was used for five wells of a 6-well plate. This yielded 0.2 mL of microbeads encapsulating 200,000 cells in each well, to which was added 2 mL of osteogenic media. Twenty-five wells were prepared, with five wells (n=5) each culturing for 1, 4, 7, 14 and 21d. Three types of microbeads totaled 75 wells for the live/dead assay. The media was changed every 2 d. The cells were stained with a live/dead kit (Invitrogen, Carlsbad, Calif.), and observed using epifluorescence microscopy (Eclipse TE-2000S, Nikon, Melville, N.Y.). Three images were taken at random locations for each sample, with five samples yielding 15 images for each type of microbeads at each time point. The live and dead cells were counted. The percentage of live cells was: P_{Live}=N_{Live}/(N_{Live}+N_{Dead}), where N_{Live}=the number of live cells, and N_{Dead}=the number of dead cells [62]. The live cell density, D_{Live}, was calculated: D_{Live}=N_{Live}/A, where A is the area of the view field for N_{Live}.

2.4. Osteogenic Differentiation of hUCMSCs

Only the type 3 microbeads were able to degrade rapidly and release the cells with enhanced proliferation. Hence, type 3 microbeads were used for measurement of osteogenic differentiation. A quantitative real-time reverse transcription polymerase chain reaction (qRT-PCR, 7900HT, Applied Biosystems, Foster City, Calif.) method was used. After culturing in the osteogenic media for 4, 7, 14 and 21d (n=5), the total cellular RNA of the cells were extracted with TRIzol reagent (Invitrogen) and reverse-transcribed into cDNA using a High-Capacity cDNA Archive kit, following previous studies [71]. TaqMan gene expression assay kits, including two pre-designed specific primers and probes, were used to measure the transcript levels of the proposed genes on human alkaline phosphatase (ALP, Hs00758162_m1), osteocalcin (OC, Hs00609452_g1), collagen type I (Hs00164004), Runx2 (Hs00231692_m1), and glyceraldehyde 3-phosphate dehydrogenase (GAPDH, Hs99999905). Relative expression for each target gene was evaluated using the 2-ΔΔCt method [65]. Ct values of target genes were normalized by the Ct of the TaqMan human housekeeping gene GAPDH to obtain the ΔCt values. The Ct of hUCMSCs cultured on tissue culture polystyrene in the control media for 1d served as the calibrator [46, 71].

2.5. hUCMSC Mineralization

The oxidized alginate-fibrin microbeads with hUCMSC encapsulation were used. After culturing for 4, 7, 14 and 21d (n=5), Alizarin Red S (ARS) staining was performed to visualize bone mineralization [66]. The adherent cells were washed with PBS, fixed with 10% formaldehyde, and stained with ARS (Millipore, Billerica, Mass.) for 5 min, which stained calcium-rich deposits made by the cells into a red color. Control wells were included with the hUCMSCs cultured in the control media. For quantification, an osteogenesis assay kit (Millipore, Billerica, Mass.) was used to extract the stained minerals and measure the Alizarin Red concentration at OD₄₀₅, following the manufacture's instructions. Time periods of up to 21d were selected because in previous studies, a great increase in calcium content during in vitro cell cultures was found between 12 d to 21d [67].

One-way and two-way ANOVA were performed to detect significant effects of the variables. Tukey's multiple comparison tests were used at a p value of 0.05.

2B. Results

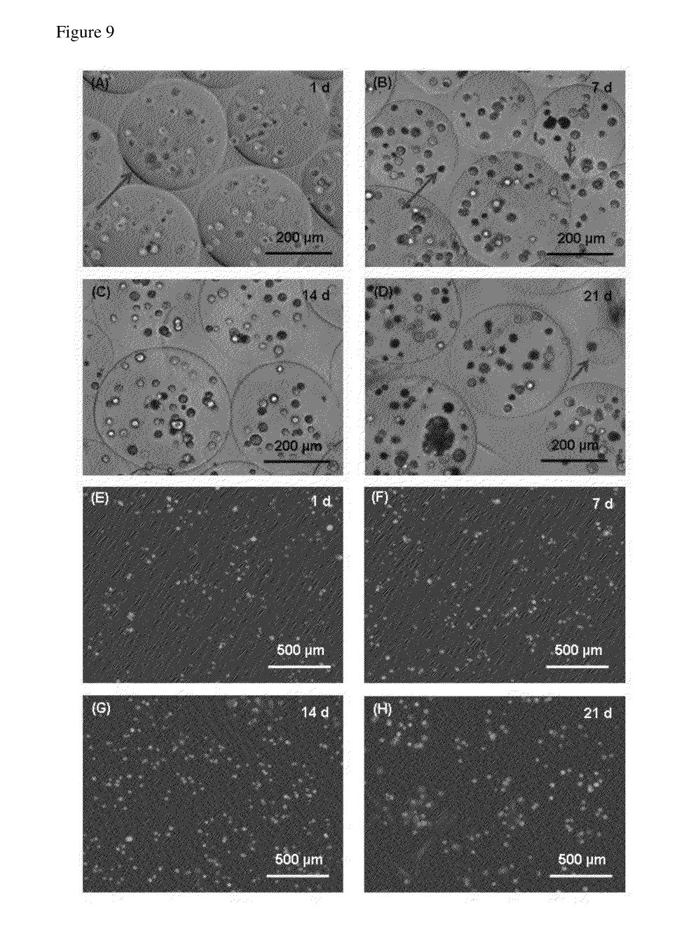

FIG. 9A-D show optical photos for alginate microbeads encapsulating hUCMSCs at 1, 7, 14 and 21d, respectively. Because the microbeads were nearly transparent and difficult to see, a blue filter was used which enhanced the contrast and clarity of the microbeads. Measurement of 100 random microbeads showed that the microbead diameter ranged from about 73 to 465 μm, with a mean of 207 μm. Typical fluorescent photos of live/dead staining are shown in FIG. 9E-H at 1, 7, 14 and 21d, respectively. The live cells appeared as small green dots. There was no noticeable microbead degradation or cell release from 1d to 21d.

Optical photos of the oxidized alginate microbeads are shown in FIG. 10A-D at 1, 7, 14 and 21d, respectively. The oxidized alginate microbeads appeared to be weaker mechanically than the non-modified alginate microbeads. Many of the microbeads appeared elongated and not perfectly spherical, likely because the gel was not stiff and the microbead spread out under its own weight. Typical fluorescent photos are shown in FIG. 10E-H at 1, 7, 14 and 21d, respectively. There is slight microbead degradation and cell release at 14 and 21d, with most cells showing as green dots but some cells showing a spreading morphology.

The microbead degradation and cell release are much more pronounced in FIG. 11 for the oxidized alginate-fibrin microbeads: (A) 1d with low magnification, (B) 1d with high magnification, (C), 4 d, (D) 7 d, (E) 14 d, and (F) 21d. Although the microbeads deviated from the spherical shape, they were intact and did not degrade at 1d. At 4 d, the microbeads started to degrade. The degradation accelerated at 7 d and numerous cells were released. The microbeads broke down at 14 d and became small pieces at 21d.

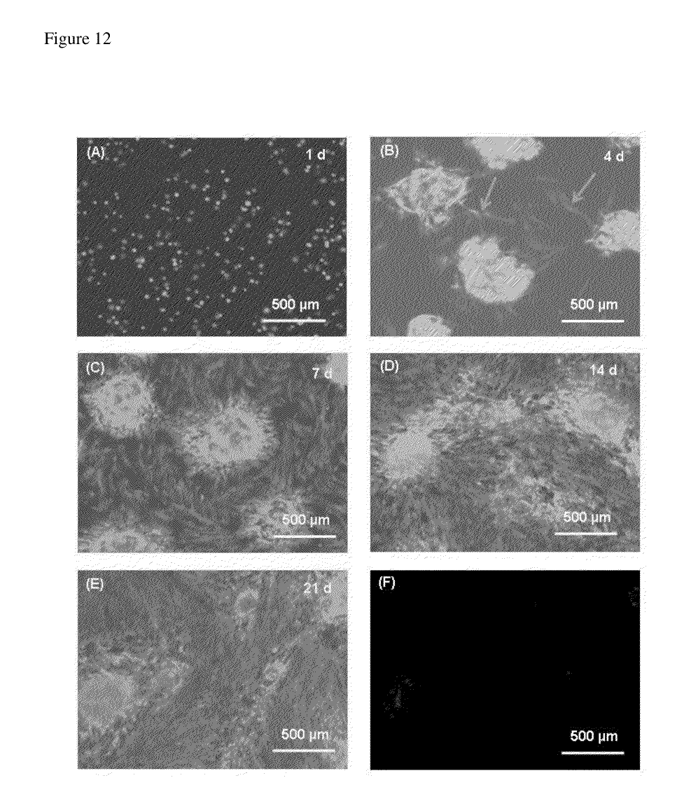

The fluorescent live/dead staining photos for the oxidized alginate-fibrin microbeads are shown in FIG. 12. At 1d, the cells appeared as small green dots, similar to those for type 1. However, at 4 d, some cells were released from the microbeads and showed a spreading morphology. The number of released cells greatly increased from 7 to 21d. The released cells proliferated, forming a confluent monolayer at 21d. In addition, the cells developed a healthy polygonal morphology, indicating that they had attached to the tissue culture polystyrene. Dead cells were stained red and were very few at all time points, with an example in (F) at 21d.

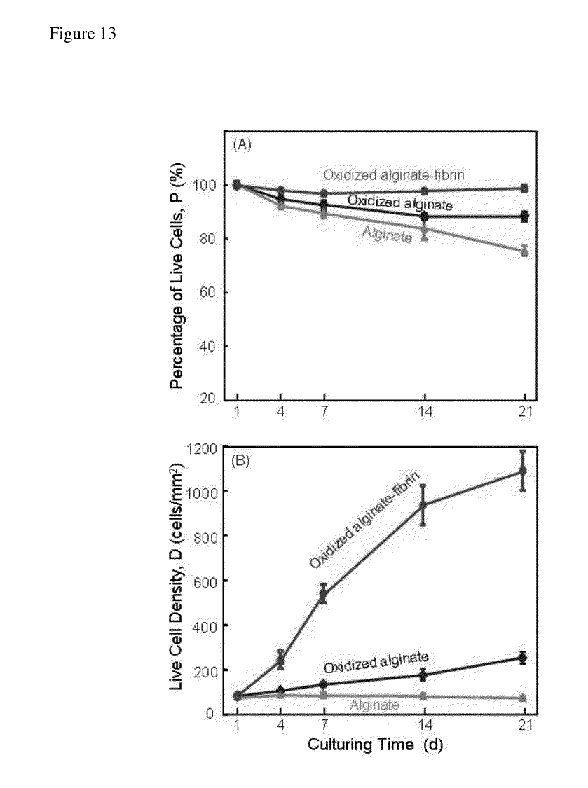

In FIG. 13A, the percentage of live cells for the oxidized alginate-fibrin group was the highest, followed by the oxidized alginate group, with the alginate group being the lowest. In (B), the number of live cells per area was nearly constant for alginate microbeads, and slightly increased with time for the oxidized alginate microbeads. In contrast, cells rapidly proliferated for the oxidized alginate-fibrin group, yielding a substantial increase in live cell density. At 21 d, the oxidized alginate-fibrin group had a live cell density that was approximately 4-fold that of the oxidized alginate group, and 15-fold that of the alginate group.

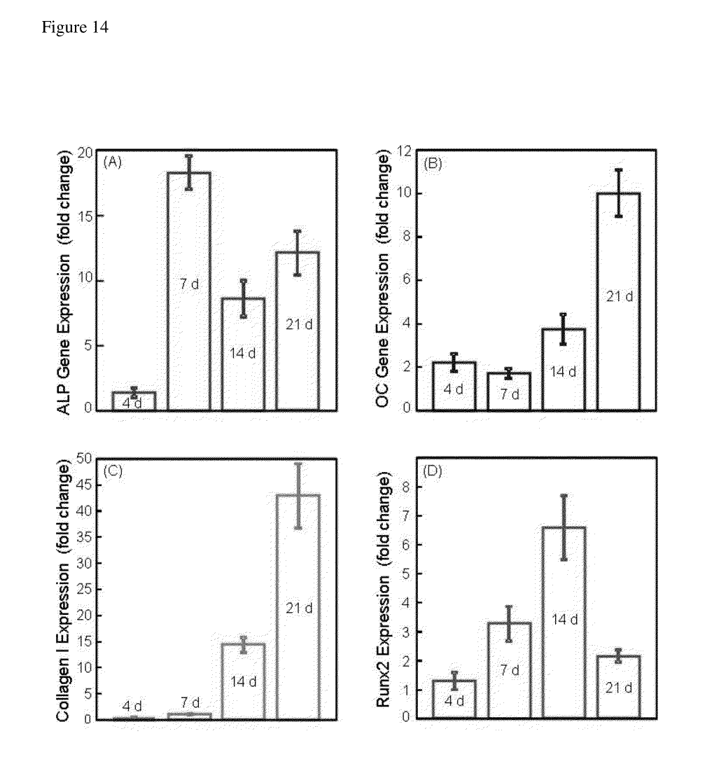

The oxidized alginate-fibrin group with successful cell release was selected for the RT-PCR experiment, and the results are plotted in FIG. 14. The ALP gene expression was minimal at 4 d, greatly increased at 7 d (p<0.05), and then decreased at 14 d and 21d. ALP at 7 d was 18-fold that at 4 d. The OC expression started to increase at 14 d and greatly increased at 21d. So did the collagen I expression. Runx2 peaked at 14 d. These data indicate that hUMSCs released from the microbeads had differentiated into the osteogenic lineage.

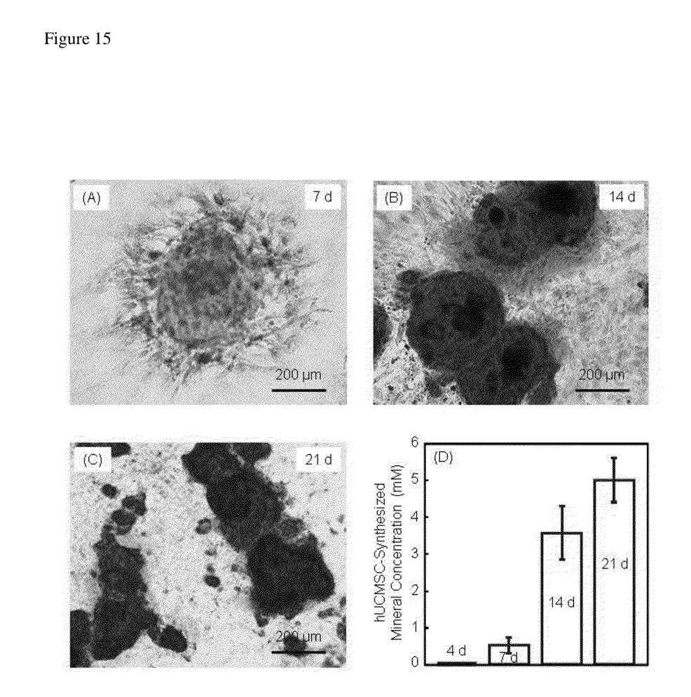

Results on hUCMSC mineralization are shown in FIG. 15. There was little mineral staining at 1 and 4 d (not shown). There was noticeable mineral synthesis in (A) at 7 d. The mineral staining greatly increased at 14 d and 21d (B and C). In (D), the mineral concentration was measured by an osteogenesis assay. The mineral synthesized by the hUCMSCs was minimal at 4 d, then increased significantly at 14 and 21d (p<0.05).

### Example 3

### 3A. Materials and Methods

3.1. Fabrication of Gas-Foaming CPC Scaffold

The CPC powder consisted of an equimolar mixture of tetracalcium phosphate (TTCP: Ca₄[PO₄]₂O) and dicalcium phosphate anhydrous (DCPA: CaHPO₄). Chitosan was used because it is biodegradable and osteoconductive [38] and can strengthen CPC [37]. Hence, the CPC liquid consisted of chitosan malate (Halosource, Redmond, Wash.) mixed with water at a chitosan/(chitosan+water) mass fraction of 15% [37]. A degradable suture fiber (Vicryl, Ethicon, Somerville, N.J.), a copolymer of glycolic and lactic acids, was cut into 3-mm filaments and used at a fiber volume fraction of 20% [71] to reinforce CPC.

A gas-foaming method was used to create macropores in CPC. Following a previous study [72], sodium hydrogen carbonate (NaHCO₃) and citric acid (citric acid monohydrate, C₆H₈O₇.H₂O) were added into CPC as the porogen. The acid-base reaction of citric acid with NaHCO₃ produced CO₂ bubbles in CPC, resulting in a macroporous scaffold [73]. NaHCO₃ was added to the CPC powder, at the following NaHCO₃/(NaHCO₃+CPC powder) mass fractions of 0%, 5%, 10%, 15%, 20%, 25%, and 30%. NaHCO₃/(NaHCO₃+CPC powder) is referred to as the mass fraction of “Foam porogen in CPC”. The corresponding amount of C₆H₈O₇.H₂O was added to the CPC liquid, to maintain a fixed NaHCO₃/(NaHCO₃+C₆H₈O₇.H₂O) mass fraction of 54.52% [73]. The CPC powder, chitosan, and porogen were sterilized in an ethylene oxide sterilizer (Andersen, Haw River, N.C.) for 12 h, and then degassed for 7 d according to the manufacturer's specifications, prior to making the specimens.

3.2. hUCMSC Culture

hUCMSCs were obtained commercially (ScienCell, Carlsbad, Calif.), which were harvested from the Wharton's Jelly in human umbilical cords of healthy babies, using an established method [11,14]. Cells were cultured in a low-glucose Dulbecco's modified Eagle's medium (DMEM), supplemented with 10% fetal bovine serum (FBS) and 1% penicillin-streptomycin (Invitrogen, Carlsbad, Calif.), and this media is termed “control media”. At 80-90% confluence, hUCMSCs were detached and passaged, and passage 4 cells were used. The osteogenic media consisted of the control media supplemented with 100 nM dexamethasone, 10 mM β-glycerophosphate, 0.05 mM ascorbic acid, and 10 nM 1α,25-Dihydroxyvitamin (Sigma) [13, 16, 71].

3.3. hUCMSC Encapsulation in Degradable Alginate-Fibrin Hydrogel Microbeads

Alginate is non-cytotoxic and can form an ionically-crosslinked network under mild conditions, and alginate microbeads were used to encapsulate cells previously [33, 34, 71]. After the CPC is set, in some circumstances and uses it can be desirable for the microbeads to quickly degrade and to release the cells so that the cells can attach to CPC and proliferate inside the CPC scaffold. Preliminary study found that adding fibrin to alginate yielded alginate-fibrin microbeads that were readily degradable and released the cells after only several days. In the present study, fibrinogen (Sigma, St. Louis, Mo.) was added to the alginate solution at a fibrinogen mass fraction of 0.12%, because preliminary study showed that this concentration resulted in microbeads that had sufficient mechanical integrity to be handled and yet could degrade and release the cells in several days.

Alginate was partially oxidized to increase its degradability. The degree of oxidation (%) was the number of oxidized uronate residues per 100 uronate units in the alginate chain, and the reaction was done at the correct stoichiometric ratio of sodium periodate to alginate to yield 7.5% oxidation, following a previous study [63]. Briefly, 1% by mass of sodium alginate (MW=75000 to 220000 g/mol, ProNova, Norway) was dissolved in distilled water. For oxidization, 1.51 mL of 0.25 M sodium periodate (Sigma) was added to 100 mL of alginate solution, which was stirred to react in the dark at room temperature. At 24 h, the oxidization reaction was stopped by adding 1 g of ethylene glycol and then 2.5 g of sodium chloride. 200 mL of ethanol was added to precipitate the product, which was then collected by centrifugation. The precipitates were re-dissolved in 100 mL of water and precipitated with 200 mL of ethanol. The second precipitates were collected and dissolved in 30 mL of water. The final product was freeze dried for 24 h, and used to make the microbeads. The oxidized alginate thus obtained was dissolved in saline at a concentration of 1% by mass. Then, fibrinogen from bovine plasma (Sigma) was added at a concentration of 0.12% and incubated at 37° C. for 2 h to yield a mixed alginate-fibrinogen solution. hUCMSCs were added at a density of 1×10⁶ cells/mL in the alginate-fibrin solution. The alginate-fibrin-cell solution was loaded into a syringe which was placed into a syringe pump and connected to a bead-generating device (Var J1, Nisco, Zurich, Switzerland), as described previously [71]. Sterile nitrogen gas was fed to the gas inlet to form a coaxial air flow to break up the alginate droplets, which were sprayed into a beaker with a solution containing 150 mL of 100 mmol/L calcium chloride plus 125 NIH units of thrombin (Sigma). Calcium chloride caused the alginate to crosslink, while the reaction between fibrinogen and thrombin generated the fibrin, thus yielding the alginate-fibrin microbeads. This produced hUCMSC-encapsulating microbeads with a mean diameter of 410±220 μm (ranging from approximately 300 μm to 600 μm).

3.4 Viability of hUCMSCs Encapsulated Inside Gas-Foaming CPC

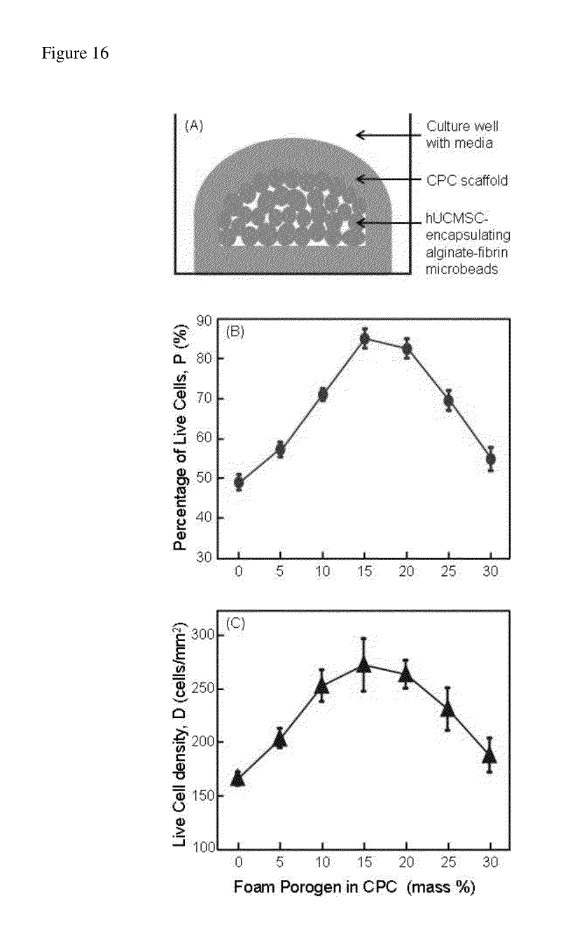

As shown in the schematic in FIG. 16A, the microbeads were completely wrapped inside CPC. First, a layer of CPC paste was placed on the well bottom to form the shape of a flat-bottomed cooking pan. Then, the hUCMSC-encapsulating microbeads were placed in the “pan”, and the top was completely covered with another layer of CPC paste. Additional CPC paste was used to seal the periphery to make sure that the two layers of CPC bonded with each other. An even more ideal situation would be to mix the microbeads randomly with the CPC paste, as would be the case in potential clinical applications. However, it was difficult to perform live/dead staining and take photos to monitor cell viability because the interior of CPC, when opened for examination, yielded a tortuous and rough surface. Therefore, the flat bottom CPC in FIG. 16A was used to provide a flat surface to do live/dead staining and to take photos for analysis. FIG. 16A was an improved setup with more strict conditions than those in previous studies [33, 34, 71], because it eliminated extra openings at the periphery in previous studies that provided media access to the cells. The cells in FIG. 16A would rely solely on the porosity in CPC for access to culture media, similar to clinical applications using a CPC paste mixed with microbeads in which the cells would rely on pores in CPC for access to physiological fluids with nutrients and oxygen. In addition, the setup in FIG. 16A allowed the examination of cell viability with the CPC setting reaction occurring on all sides, and the cell release from the microbeads and cell attachment on the CPC bottom surface, instead of on the tissue culture polystyrene well.

Two groups of specimens were tested. The purpose of group 1 was to investigate the effect of the gas-foaming porogen mass fraction in CPC on cell viability inside CPC. As described in Section 3.1, the following NaHCO₃/(NaHCO₃+CPC powder) mass fractions were used: 0%, 5%, 10%, 15%, 20%, 25%, and 30%. Each CPC paste and microbeads were placed as shown in FIG. 16A, at a microbeads/(CPC+microbead) volume fraction of 50%. After CPC hardening at 37° C. for 30 minutes, 5 mL of osteogenic media was added to each well to submerge the construct. After 1d, the CPC construct was opened and live/dead stained (Molecular Probes, Eugene, Oreg.), and the cells on the bottom CPC surface was examined. Live cells were stained green and dead cells were stained red. The cells were observed using epifluorescence microscopy (Eclipse TE-20005, Nikon, Melville, N.Y.). Three random images per disk yielded 15 images for each material. The cells were counted, and the percentage of live cells was measured as P=N_{LIVE}/(N_{LIVE}+N_{DEAD}), where N_{LIVE} is the number of live cells, and N_{DEAD} is the number of dead cells [70]. The live cell density was also measured: D=N_{LIVE}/A, where A was the area of the image where N_{LIVE} was measured.

The purpose of group 2 was to examine the effect of culture time from 1d to 21d on cell viability inside CPC. The results from group 1 showed that CPC with 15% foaming porogen yielded the best cell viability, hence group 2 used 15% foaming porogen. The culture times were: 1d, 7 d, 14 d, and 21d. At each time period, the CPC construct was opened and the cell live/dead staining was performed and measured, in the same manner as for group 1.

3.5. Measurement of Gas-Foaming CPC Porosity and Mechanical Properties

Group 1 showed that the cell viability was high at foam porogen mass fractions of 10%, 15% and 20% in CPC. Cell viability was lower at 0%, 5%, 25% and 30% porogen. Hence, the porosity and mechanical properties were measured for scaffolds at foam porogen of 10%, 15% and 20%, with 0% as control. To measure porosity, the CPC specimens were dried in a vacuum oven at 60° C. for 24 h. The density of CPC was measured using the specimen weight divided by volume [69]. The volume was calculated by the specimen dimensions measured with a micrometer, with each dimension being the average of three locations along the specimen. Six specimens were measured at each porogen content. Following a previous study [76], the pore volume fraction of CPC-chitosan specimens was obtained by: p=(d_{HA+CN}−d)/d_{HA+CN}, where p is porosity, and d is the measured density. The density of fully-dense hydroxyapatite d_{HA}=3.14 g/cm^{3 [}75]. The CPC contained 15% chitosan, and the fully-dense hydroxyapatite-chitosan composite density d_{HA+CN}=2.82 g/cm³, as obtained in a previous study [76]. The experimentally-measured density d leads to the calculation of the porosity p. A scanning electron microscope (SEM, FEI Quanta 200, Hillsboro, Oreg.) was used to examine the pore morphology in specimens sputter coated with gold.

A three-point flexural test was used to fracture the bar specimens on a Universal Testing Machine (MTS, Eden Prairie, Minn.) using a span of 20 mm at a crosshead speed of 1 mm/min [44]. Flexural strength, S=3FₘₐₓL/(2bh²), where Fₘₐₓ is the maximum load on the load-displacement (F-a) curve, L is span, b is specimen width and h is thickness. Elastic modulus E=(F/a) (L³/[4bh³]). Work-of-fracture (toughness) was the area under the F-d curve divided by the specimen's cross-sectional area [40].

One-way and two-way ANOVA were performed to detect significant effects of the variables. Tukey's multiple comparison tests were used at a p value of 0.05.

### 3B. Results

FIG. 16 shows the effect of foam porogen mass fraction on the viability of the encapsulated hUCMSCs: (B) Percentage of live cells P, and (C) live cell density D, after 1d. For CPC without porogen, P was only 49%, with a lot of dead cells. This indicates that the porosity in CPC without foam porogen was not sufficient to provide media access to the cells inside CPC. Increasing the porogen mass fraction significantly (p<0.05) increased P, reaching 86% at 15% porogen. When the foam porogen was further increased, P started to decrease. In (C), the number of live cells was 160 cells/mm² in CPC without porogen, increased to 270 cells/mm² at 15% porogen, and then started to decrease when the porogen was further increased.

Typical live/dead stained photos are shown in FIG. 17, where the live cells were stained green and dead cells were stained red. (A) and (B) showed that inside CPC without porogen, there was a large number of dead cells. The number of dead cells dramatically decreased when the porogen was increased to 10%, 15% and 20%, as shown in (C) to (H). Meanwhile, the live cell density increased. However, when the foam porogen was further increased to 30%, the number of live cells decreased in (I) and dead cells increased in (J).

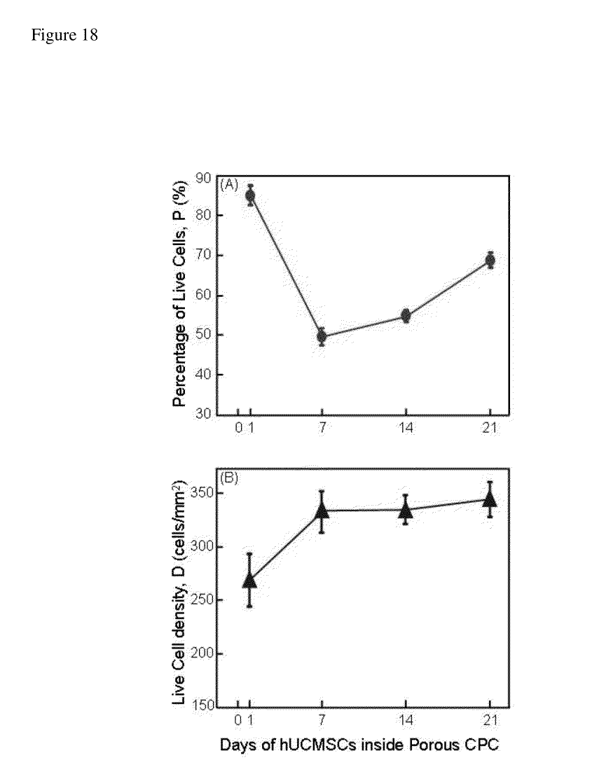

FIG. 18 shows the effect of culture time from 1d to 21d on the encapsulated hUCMSC viability inside CPC with 15% foam porogen. The percentage of live cells decreased to 49% at 7 d, and then started to increase, reaching 69% at 21d. The live cell density increased over time, from 270 cells/mm² at 1d to 350 cells/mm² at 21d (p<0.05).

The alginate-fibrin microbeads degraded and released the hUCMSCs inside CPC. When encapsulated in hydrogel, a live cell was seen as a green dot in the fluorescent photo, examples of which are shown in FIG. 17 at 1d. At 7 d, when the CPC bottom surface (FIG. 16A) was observed, cells in some areas were seen as green dots, indicating that they were still encapsulated in the fragments of the degraded microbeads. However, there were other areas where the cells showed a spreading morphology with an elongated spindle shape, typical of free (not encapsulated) MSCs attaching to a substrate. An example of this is shown in FIG. 19A at 7 d. The spread and elongated morphology was shown in FIG. 19B at 14 d. At 21d (FIG. 19C), the number of released and attached cells greatly increased, indicated by a high density of cells with a spreading morphology and spindle shapes. In contrast, cells in alginate microbeads without fibrin appeared as green dots in FIG. 19D, with no sign of cell spreading at 21d. Furthermore, the alginate microbeads without fibrin could be collected with mechanical integrity after culturing for 21d. However, the alginate-fibrin microbeads were degraded into fragments and became invisible even in the microscope after 7 d, and no integral microbeads could be found or collected. These results indicate that the alginate-fibrin microbeads could be degraded and the hUCMSCs could be released inside CPC after 7 d, and the released cells showed a healthy spreading morphology with good viability after 21d.

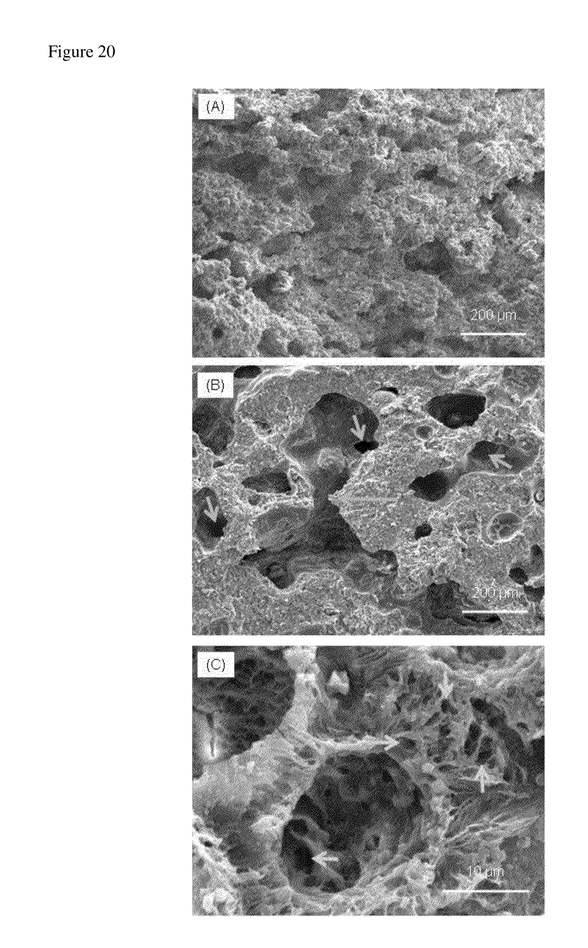

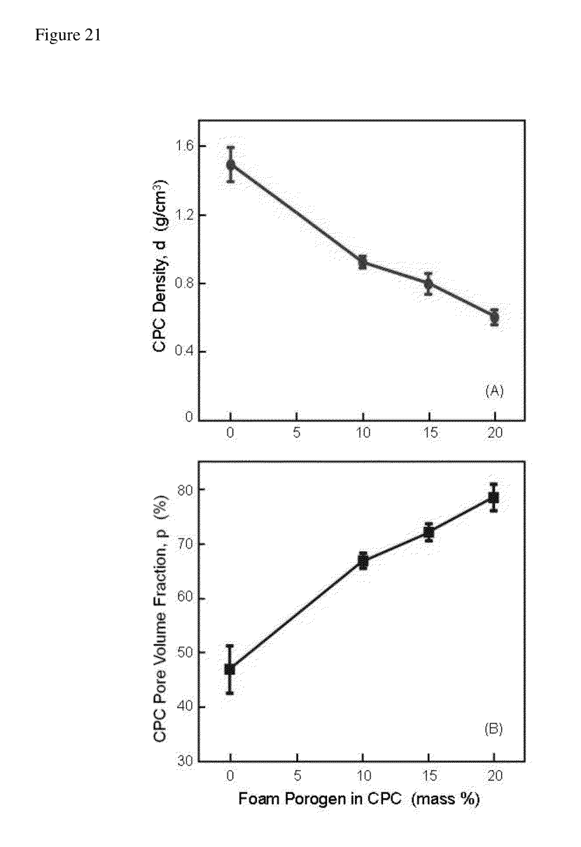

FIG. 20 shows typical porosity in CPC with (A) 0%, (B) 10%, and (C) 20% foam porogens. The macropores had sizes of about 100 to 400 p.m. The pores were interconnected. The long arrow in (B) indicates the interconnection between two pores. The short arrows indicate opening fenestrations inside pores leading to the next pores. A higher magnification in (C) showed numerous micropores on the macropore wall contributing to the interconnectivity. Regardless of the porogen content, the CPC matrix was full of micropores with submicron to several microns in sizes. FIG. 21 plots (A) the density, and (B) the porosity of CPC vs. porogen content. The density decreased from 1.5 g/cm³ to 0.6 g/cm³ when the porogen in CPC was increased from 0% to 20%. The respective pore volume fraction in CPC was increased from 46.8% to 78.4%.

The mechanical properties of CPC vs. foam porogen content are plotted in FIG. 22. The strength was 8.6 MPa at 0% porogen, but decreased to 0.4 MPa at 20% porogen. Fiber reinforcement greatly improved the strength. The strength was increased to 19.9 MPa at 0% porogen, 3.8 MPa at 15% porogen, and 2.2 MPa at 20% porogen. Work-of-fracture of porous CPC was also substantially increased via fiber reinforcement. The elastic modulus showed a decreasing trend with increasing the foam porogen in CPC.

### INCORPORATION BY REFERENCE

Throughout this application, various publications, patents, and/or patent applications are referenced in order to more fully describe the state of the art to which this invention pertains. The disclosures of these publications, patents, and/or patent applications are herein incorporated by reference in their entireties to the same extent as if each independent publication, patent, and/or patent application was specifically and individually indicated to be incorporated by reference.

### Other Embodiments

It will be apparent to those skilled in the art that various modifications and variations can be made in the present invention without departing from the scope or spirit of the invention. Other embodiments of the invention will be apparent to those skilled in the art from consideration of the specification and practice of the invention disclosed herein. It is intended that the specification and examples be considered as exemplary only, with a true scope and spirit of the invention being indicated by the following claims.

### REFERENCES

[1] Praemer A, Furner S, Rice D P. Musculoskeletal conditions in the United States. Rosemont, Ill.: Amer Acad Orthop Surg; 1999. Chapter 1.
[2] Laurencin C T, Ambrosio A M A, Borden M D, Cooper J A. Tissue engineering: Orthopedic applications. Annual Rev Biomed Eng 1999; 1:19-46.
[3] Sikavitsas V I, Bancroft G N, Holtorf H L, Jansen J A, Mikos A G. Mineralized matrix deposition by marrow stromal osteoblasts in 3D perfusion culture increases with increasing fluid shear forces. Proc Natl Acad Sci USA 2003; 100:14683-14688.
[4] Mauney J, Volloch V, Kaplan D L. Matrix-mediated retention of osteogenic differentiation potential by human adult bone marrow stromal cells during ex vivo expansion. Biomaterials 2004; 25:3233-3243.
[5] Yao J, Radin S, Reilly G, Leboy P S, Ducheyne P. Solution-mediated effect of bioactive glass in poly (lactic-co-glycolic acid)-bioactive glass composites on osteogenesis of marrow stromal cells. J Biomed Mater Res 2005; 75A:794-801.
[6] Mao J J, Giannobile W V, Helms J A, Hollister S J, Krebsbach P H, Longaker M T et al. Craniofacial tissue engineering by stem cells. J Dent Res 2006; 85:966-979.
[7] Datta N, Pham Q P, Sharma U, Sikavitsas V I, Jansen J A, Mikos A G. In vitro generated extracellular matrix and fluid shear stress synergistically enhance 3D osteoblastic differentiation. Proceed Natl Acad Sci USA, 2006; 103:2488-2493.
[8] Silva G A, Coutinho O P, Ducheyne P, Reis R L. Materials in particulate form for tissue engineering, Part 2: Applications in bone (review). J Tissue Eng Regen Med 2007; 1:97-109.
[9] Mao J J, Vunjak-Novakovic G, Mikos A G, Atala A. Regenerative medicine: Translational approaches and tissue engineering. Artech House, Boston and London, 2007.
[10] Kretlow J D, Young S, Klouda L, Wong M, Mikos A G. Injectable biomaterials for regenerating complex craniofacial tissues. Adv Mater 2009; 21:3368-3393.
[11] Wang H S, Hung S C, Peng S T. Mesenchymal stem cells in the Wharton's jelly of the human umbilical cord. Stem Cells 2004; 22:1330-1337.
[12] Can A, Karahuseyinoglu S. Concise review: Human umbilical cord stroma with regard to the source of fetus-derived stem cells. Stem Cells 2007; 25:2886-2895.
[13] Baksh D, Yao R, Tuan R S. Comparison of proliferative and multilineage differentiation potential of human mesenchymal stem cells derived from umbilical cord and bone marrow. Stem Cells 2007; 25:1384-1392.
[14] Bailey M M, Wang L, Bode C J, Mitchell K E, Detamore M S. A comparison of human umbilical cord matrix stem cells and temporomandibular joint condylar chondrocytes for tissue engineering temporomandibular joint condylar cartilage. Tissue Eng 2007; 13:2003-2010.
[15] Karahuseyinoglu S, Kocaefe C, Balci D, Erdemli E, Can A. Functional structure of adipocytes differentiated from human umbilical cord stroma-derived stem cells. Stem Cells 2008; 26:682-691.
[16] Wang L, Singh M, Bonewald L F, Detamore M S. Signaling strategies for osteogenic differentiation of human umbilical cord mesenchymal stromal cells for 3D bone tissue engineering. J Tissue Eng Regen Med 2009; 3:398-404.
[17] Zhao L, Burguera E F, Xu H H K, Amin N, Ryou H, Arola D D. Fatigue and human umbilical cord stem cell seeding characteristics of calcium phosphate-chitosan-biodegradable fiber scaffolds. Biomaterials 2010; 31:840-847.
[18] Jäger M, Degistirici O, Knipper A, Fischer J, Sager M, Krauspe R. Bone healing and migration of cord blood-derived stem cells into a critical size femoral defect after xenotransplantation. J Bone Miner Res 2007; 22:1224-1233.
[19] Zhao L, Weir M D, Xu H H K. Human umbilical cord stem cell encapsulation in calcium phosphate scaffolds for bone engineering. Biomaterials 2010; 31:3848-3857.
[20] Ducheyne P, Qiu Q. Bioactive ceramics: the effect of surface reactivity on bone formation and bone cell function. Biomaterials 1999; 20:2287-2303.
[21] Pilliar R M, Filiaggi M J, Wells J D, Grynpas M D, Kandel R A. Porous calcium polyphosphate scaffolds for bone substitute applications—in vitro characterization. Biomaterials 2001; 22:963-972.
[22] Foppiano S, Marshall S J, Marshall G W, Saiz E, Tomsia A P. The influence of novel bioactive glasses on in vitro osteoblast behavior. J Biomed Mater Res A 2004; 71:242-249.
[23] Reilly G C, Radin S, Chen A T, Ducheyne P. Differential alkaline phosphatase responses of rat and human bone marrow derived mesenchymal stem cells to 45S5 bioactive glass. Biomaterials 2007; 28:4091-4097.
[24] Deville S, Saiz E, Nalla R K, Tomsia A P. Freezing as a path to build complex composites. Science 2006; 311:515-518.
[25] Drury J L, Mooney D J. Review. Hydrogels for tissue engineering: scaffold design variables and applications. Biomaterials 2003; 24:4337-4351.
[26] Brown W E, Chow L C. A new calcium phosphate water setting cement. In: Cements research progress. Brown P W, editor. Westerville, Ohio: Am Ceram Soc. p. 352-379, 1986.
[27] Barralet J E, Gaunt T, Wright A J, Gibson I R, Knowles J C. Effect of porosity reduction by compaction on compressive strength and microstructure of calcium phosphate cement. J Biomed Mater Res B 2002; 63:1-9.
[28] Bohner M, Baroud G. Injectability of calcium phosphate pastes. Biomaterials 2005; 26:1553-1563.
[29] Link D P, van den Dolder J, van den Beucken J J, Wolke J G, Mikos A G, Jansen J A. Bone response and mechanical strength of rabbit femoral defects filled with injectable CaP cements containing TGF-β1 loaded gelatin microspheres. Biomaterials 2008; 29:675-682.
[30] Shindo M L, Costantino P D, Friedman C D, Chow L C. Facial skeletal augmentation using hydroxyapatite cement. Arch Otolaryngol Head Neck Surg 1993; 119:185-190.
[31] Friedman C D, Costantino P D, Takagi S, Chow L C. BoneSource hydroxyapatite cement: a novel biomaterial for craniofacial skeletal tissue engineering and reconstruction. J Biomed Mater Res B 1998; 43:428-432.
[32] Xu H H K, Chow L C, Takagi S, Eichmiller F C. Self-hardening calcium phosphate materials with high resistance to fracture, controlled strength histories and tailored macropore formation rates. U.S. Pat. No. 6,955,716, 2005.
[33] Simon C G, Jr., Guthrie W F, Wang F W. Cell seeding into calcium phosphate cement. J Biomed Mater Res A 2004; 68:628-639.
[34] Weir M D, Xu H H K, Simon C G. Strong calcium phosphate cement-chitosan-mesh construct containing cell-encapsulating hydrogel beads for bone tissue engineering. J Biomed Mater Res 2006; 77A:487-496.
[35] Xu H H K, Weir M. Three-Dimensional Cell Protector/Pore Architecture Formation for Bone and Tissue Constructs. U.S. patent Ser. No. 11/132,028, allowed Nov. 25, 2009.
[36] Koch S, Schwinger C, Kressler J, Heinzen C, Rainov N G. Alginate encapsulation of genetically engineered mammalian cells: comparison of production devices, methods and microcapsule characteristics. J Microencapsul 2003; 20:303-316.
[37] Xu H H K, Takagi S, Quinn J B, Chow L C. Fast-setting and anti-washout calcium phosphate scaffolds with high strength and controlled macropore formation rates. J Biomed Mater Res A 2004; 68:725-734.
[38] Muzzarelli R A A, Biagini G, Bellardini M, Simonelli L, Castaldini C, Fraatto G. Osteoconduction exerted by methylpyrrolidinone chitosan in dental surgery. Biomaterials 1993; 14:39-43.
[39] Xu H H K, Simon C G. Fast setting calcium phosphate-chitosan scaffold: mechanical properties and biocompatibility. Biomaterials 2005; 26:1337-1348.
[40] Xu H H K, Quinn J B. Calcium phosphate cement containing resorbable fibers for short-term reinforcement and macroporosity. Biomaterials 2002; 23:193-202.
[41] Burguera E F, Xu H H K, Weir M D. Injectable and rapid-setting calcium phosphate bone cement with dicalcium phosphate dihydrate. J Biomed Mater Res 2006; 77B:126-134.
[42] Xu H H K, Weir M D, Burguera E F, Fraser A M. Injectable and macroporous calcium phosphate cement scaffold. Biomaterials 2006; 27:4279-4287.
[43] Bai B, Jazrawi L M, Kummer F J, Spivak J M. The use of an injectable, biodegradable calcium phosphate bone substitute for the prophylactic augmentation of osteoporotic vertebrae and the management of vertebral compression fractures. Spine 1999; 24:1521-1526.
[44] American Society for Testing and Materials. ASTM D 790-03: Standard test methods for flexural properties of unreinforced and reinforced plastic and electrical insulating materials. West Conshohocken, Pa.: ASTM International, 2004.
[45] Moreau J L, Xu H H K. Mesenchymal stem cell proliferation and differentiation on an injectable calcium phosphate-chitosan composite scaffold. Biomaterials 2009; 30:2675-2682.
[46] Kim K, Dean D, Mikos A G, Fisher J P. Effect of initial cell seeding density on early osteogenic signal expression of rat bone marrow stromal cells cultured on cross-linked poly(propylene fumarate) disks. Biomacromolecules 2009; 10:1810-1817.
[47] Orii H, Sotome S, Chen J, Wang J, Shinomiya K. Beta-tricalcium phosphate (beta-TCP) graft combined with bone marrow stromal cells (MSCs) for posterolateral spine fusion. J Med Dent Sci 2005; 52:51-57.
[48] Sun H, Wu C, Dai K, Chang J, Tang T. Proliferation and osteoblastic differentiation of human bone marrow-derived stromal cells on akermanite-bioactive ceramics. Biomaterials 2006; 27:5651-5657.
[49] Nair M B, Bernhardt A, Lode A, Heinemann C, Thieme S, Hanke T, Varma H, Gelinsky M, John A. A bioactive triphasic ceramic-coated hydroxyapatite promotes proliferation and osteogenic differentiation of human bone marrow stromal cells. J Biomed Mater Res 2009; 90A:533-542.
[50] Detsch R, Uhl F, Deisinger U, Ziegler G. 3D-Cultivation of bone marrow stromal cells on hydroxyapatite scaffolds fabricated by dispense-plotting and negative mould technique. J Mater Sci: Mater Med 2008; 19:1491-1496.
[51] Ginebra M P, Rilliard A, Fernández E, Elvira C, Román JS, Planell J A. Mechanical and rheological improvement of a calcium phosphate cement by the addition of a polymeric drug. J Biomed Mater Res 2001; 57:113-118.
[52] Sarda S, Fernandez E, Nilsson M, Balcells M, Planell J A. Kinetic study of citric acid influence on calcium phosphate bone cement as water-reducing agent. J Biomed Mater Res 2002; 61:653-659.
[53] Baroud G, Wu J Z, Bohner M, Sponagel S, Steffen T. How to determine the permeability for cement infiltration of osteoporotic cancellous bone. Med Eng Phys 2003; 25:283-288.
[54] Gbureck U, Barralet J E, Spatz K, Grover L M, Thull R. Ionic modification of calcium phosphate cement viscosity: Part I: hypodermic injection and strength improvement of apatite cement. Biomaterials 2004; 25:2187-2195.
[55] Damien C J, Parsons J R. Bone graft and bone graft substitutes: A review of current technology and applications. J Appl Biomater 1991; 2:187-208.
[56] Shi X, Sitharaman B, Pham Q P, Liang F, Wu K, Billups W E et al. Fabrication of porous ultra-short single-walled carbon nanotube nanocomposite scaffolds for bone tissue engineering. Biomaterials 2007; 28:4078-4090.
[57] Drury J L, Dennis R G, Mooney D J. The tensile properties of alginate hydrogels. Biomaterials 2004; 25:3187-3199.
[58] Kuo C K, Ma P X. Ionically crosslinked alginate hydrogels as scaffolds for tissue engineering: Part I. Structure, gelation rate and mechanical properties. Biomaterials 2001; 22:511-521.
[59] O'Kelly K, Tancred D, McCormack B, Can A. A quantitative technique for comparing synthetic porous hydroxyapatite structure and cancellous bone. J Mater Sci: Mater in Med 1996; 7:207-213.
[60] Benoit D S W, Nuttelman C R, Collins S D, Anseth K S. Synthesis and characterization of a fluvastatin-releasing hydrogel delivery system to modulate hMSC differentiation and function for bone regeneration. Biomaterials 2006; 27:6102-6110.
[61] Leach J K, Kaigler D, Wang Z, Krebsbach P H, Mooney D J. Coating of VEGF-releasing scaffolds with bioactive glass for angiogenesis and bone regeneration. Biomaterials 2006; 27; 3249-3255.
[62] Xu H H K, Zhao L, Detamore M S, Takagi S, Chow L C. Umbilical cord stem cell seeding on fast-resorbable calcium phosphate bone cement. Tissue Engineering Part A 2010; 16:2743-2753.
[63] Bouhadir K H, Lee K Y, Alsberg E, Damm K L, Anderson K W, Mooney D J. Degradation of partially oxidized alginate and its potential application for tissue engineering. Biotechnology Progress 2001; 17:945-950.
[64] Kong H J, Smith M K, Mooney D J. Designing alginate hydrogels to maintain viability of immobilized cells. Biomaterials 2003; 24:4023-4029.
[65] Livak K J, Schmittgen T D. Analysis of relative gene expression data using real-time quantitative PCR and the 2-ΔΔCt Method. Methods 2001; 25:402-408.
[66] Varghese S, Hwang N S, Ferran A, Hillel A, Theprungsirikul P, Canver A C, Zhang Z, Gearhart J, Elisseeff J. Engineering musculoskeletal tissues with human embryonic germ cell derivatives. Stem Cells 2010; 28:765-774.
[67] Wang Y H, Liu Y, Maye P, Rowe D W. Examination of mineralized nodule formation in living osteoblastic cultures using fluorescent dyes. Biotechnol Prog 2006; 22:1697-1701.
[68] Xu H H K, Zhao L, Weir M D. Stem cell-calcium phosphate constructs for bone engineering. J Dent Res 2010; 89:1482-1488.
[69] Xu H H K, Quinn J B, Takagi S, Chow L C, Eichmiller F C. 2001, Strong and macroporous calcium phosphate cement: Effects of porosity and fiber reinforcement. J Biomed Mater Res A 57: 457-466.
[70] Xu H H K, Zhao L, Detamore M S, Takagi S, Chow L C. 2010, Umbilical cord stem cell seeding on fast-resorbable calcium phosphate bone cement. Tissue Engineering A 16: 2743-2753.
[71] Zhao L, Weir M D, Xu H H K. An injectable calcium phosphate—alginate hydrogel—umbilical cord mesenchymal stem cell paste for bone tissue engineering. Biomaterials 2010; 31:6502-6510.
[72] Hesaraki S, Mortarzadeh F, Sharifi D. 2007, Formation of interconnected macropores in apatitic calcium phosphate bone cement with the use of an effervescent additive. J Biomed Mater Res A 83: 80-87.
[73] Hesaraki S, Zamanian A, Mortarzadeh F. 2008, The influence of the acidic component of the gas-foaming porogen used in preparing an injectable porous calcium phosphate cement on its properties: Acetic acid versus citric acid. J Biomed Mater Res B 86: 208-216.
[74] Salinas C N, Cole B B, Kasko A M, Anseth K S. 2007, Chondrogenic differentiation potential of human mesenchymal stem cells photoencapsulated within poly(ethylene glycol)-arginine-glycine-aspartic acid-serine thiol-methacrylate mixed-mode networks. Tissue Eng 13: 1025-1034.
[75] Takagi S, Chow L C. 2001, Formation of macropores in calcium phosphate cement implants. J Mater Sci: Mater Med 12: 135-139.
[76] Zhang Y, Xu H H K, Takagi S, Chow L C. 2006, In-situ hardening hydroxyapatite-based scaffold for bone repair. J Mater Sci: Mater in Medicine 17: 437-445.

What is claimed is:

1. A bone paste comprising a calcium phosphate cement and microbeads, wherein the microbeads encapsulate cells.

2. The bone paste of claim 1, wherein the microbeads are hydrogel microbeads.

3. The bone paste of claim 1, wherein the microbeads are comprised of alginate, partially oxidized alginate, oxidized alginate, alginate-fibrin, partially oxidized alginate-fibrin, oxidized alginate-fibrin, poly(ethylene glycol diacrylate), poly(ethylene glycol)-anhydride dimethacrylate, gelatin, chemically cross-linked polymers, ionically cross-linked polymers, heat-polymerized polymers, or photopolymerized polymers.

4. The bone paste of claim 1, wherein the microbeads are alginate-fibrin microbeads.

5. The bone paste of claim 4, wherein the alginate-fibrin microbeads comprise a fibrinogen mass fraction of from about 0.05% to about 1%.

6. The bone paste of claim 4, wherein the alginate-fibrin microbeads comprise a fibrinogen mass fraction of about 0.12%.

7. The bone paste of any one of claims 1-6, wherein the microbeads are present in a volume of about 40 to 60%.

8. The bone paste of any one of claims 1-6, wherein the microbeads have an average diameter of less than about 2 millimeters.

9. The bone paste of any one of claims 1-6, wherein the cells are stem cells.

10. The bone paste of any one of claims 1-6, wherein the cells are selected from the group consisting of human umbilical cord mesenchymal stem cells, bone marrow stem cells, embryonic stem cells, pluripotent stem cells, induced pluripotent stem cells, multipotent stem cells, progenitor cells, and osteoblasts.

11. The bone paste of any one of claims 1-6, wherein the calcium phosphate cement comprises one or more ingredients selected from the group consisting of tetracalcium phosphate (TTCP) (Ca₄(PO₄)₂O), dicalcium phosphate anhydrous (DCPA) (CaHPO₄), dicalcium phosphate dihydrate (CaHPO₄.2H₂O), tricalcium phosphate (Ca₃[PO₄]₂), α-tricalcium phosphate (α-Ca₃(PO₄)₂), β-tricalcium phosphate (β-Ca₃(PO₄)₂), octacalcium phosphate (Ca₈H₂(PO₄)₆.5H₂O), amorphous calcium phosphate (Ca₃(PO₄)₂), calcium carbonate (CaCO₃), calcium hydroxide (Ca[OH]₂), and hydroxyapatite (Ca₁₀(PO₄)₆(OH)₂), and mixtures thereof.

12. The bone paste of any one of claims 1-6, wherein the calcium phosphate cement comprises tetracalcium phosphate and dicalcium phosphate anhydrous.

13. The bone paste of claim 12, wherein the calcium phosphate cement comprises a molar ratio of tetracalcium phosphate to dicalcium phosphate anhydrous of about 1:5 to about 5:1.

14. The bone paste of claim 12, wherein the calcium phosphate cement comprises a molar ratio of tetracalcium phosphate to dicalcium phosphate anhydrous of about 1:3 to about 1:1.

15. The bone paste of claim 12, wherein the calcium phosphate cement comprises an approximately 1:1 molar ratio of tetracalcium phosphate to dicalcium phosphate anhydrous.

16. The bone paste of any one of claims 1-6, wherein the calcium phosphate cement further comprises chitosan.

17. The bone paste of any one of claims 1-6, wherein the calcium phosphate cement further comprises fibers.

18. The bone paste of claim 17, wherein the fibers are degradable.

19. The bone paste of any one of claims 1-6, wherein the calcium phosphate cement further comprises a porogen.

20. The bone paste of claim 19, wherein the porogen is NaHCO₃ and citric acid.

21. The bone paste of claim 20, wherein the mass fraction of NaHCO₃ is from about 5% to about 30%, and the mass fraction of citric acid is from about 50% to about 60%.

22. The bone paste of any one of claims 1-6, wherein the bone paste further comprises a bioactive agent.

23. The bone paste of any one of claims 1-6, wherein the bone paste is injectable.

24. A bone paste, comprising:
(a) calcium phosphate cement, wherein the calcium phosphate cement comprises a porogen;
(b) alginate-fibrin microbeads, wherein the microbeads encapsulate cells;
(c) chitosan; and
(d) degradable fibers.

25. The bone paste of claim 24, wherein the alginate-fibrin microbeads comprise a fibrinogen mass fraction of from about 0.05% to about 1%.

26. The bone paste of claim 24, wherein the alginate-fibrin microbeads comprise a fibrinogen mass fraction of about 0.12%.

27. The bone paste of any one of claims 24-26, wherein the microbeads have an average diameter of less than about 2 millimeters.

28. The bone paste of any one of claims 24-26, wherein the microbeads are present in a volume of about 40 to 60%.

29. The bone paste of any one of claims 24-26, wherein the cells are stem cells.

30. The bone paste of any one of claims 24-26, wherein the cells are selected from the group consisting of human umbilical cord mesenchymal stem cells, bone marrow stem cells, embryonic stem cells, pluripotent stem cells, induced pluripotent stem cells, multipotent stem cells, progenitor cells, and osteoblasts.

31. The bone paste of any one of claims 24-26, wherein the calcium phosphate cement comprises one or more ingredients selected from the group consisting of tetracalcium phosphate (TTCP) (Ca₄(PO₄)₂O), dicalcium phosphate anhydrous (DCPA) (CaHPO₄), dicalcium phosphate dihydrate (CaHPO₄.2H₂O), tricalcium phosphate (Ca₃[PO₄]₂), α-tricalcium phosphate (α-Ca₃(PO₄)₂), β-tricalcium phosphate (β-Ca₃(PO₄)₂), octacalcium phosphate (Ca₈H₂(PO₄)₆. 5H₂O), amorphous calcium phosphate (Ca₃(PO₄)₂), calcium carbonate (CaCO₃), calcium hydroxide (Ca[OH]₂), and hydroxyapatite (Ca₁₀(PO₄)₆(OH)₂), and mixtures thereof.

32. The bone paste of any one of claims 24-26, wherein the calcium phosphate cement comprises tetracalcium phosphate and dicalcium phosphate anhydrous.

33. The bone paste of claim 32, wherein the calcium phosphate cement comprises a molar ratio of tetracalcium phosphate to dicalcium phosphate anhydrous of about 1:5 to about 5:1.

34. The bone paste of claim 32, wherein the calcium phosphate cement comprises a molar ratio of tetracalcium phosphate to dicalcium phosphate anhydrous of about 1:3 to about 1:1.

35. The bone paste of claim 32, wherein the calcium phosphate cement comprises an approximately 1:1 molar ratio of tetracalcium phosphate to dicalcium phosphate anhydrous.

36. The bone paste of any one of claims 24-26, wherein the porogen is NaHCO₃ and citric acid.

37. The bone paste of any one of claim 36, wherein the mass fractions of NaHCO₃ is from about 5% to about 30%, and the mass fraction of citric acid is from about 50% to about 60%.

38. The bone paste of any one of claims 24-26, wherein the bone paste further comprises a bioactive agent.

39. The bone paste of any one of claims 24-26, wherein the bone paste is injectable

40. The bone paste of claim 1 or 24, wherein at least 90% of a sample of the bone paste can be injected using an injection force of about 100 Newtons or less.

41. The bone paste of claim 1 or 24, wherein viability of the cells after injection of the bone paste is at least about 65% of the viability of cells in non-injected bone paste.

42. The bone paste of claim 1 or 24, wherein the bone paste, after hardening, has a flexural strength substantially similar to that of natural cancellous bone.

43. A method of repairing or remodeling a bone, comprising administering to a bone an effective amount of the bone paste of claim 1 or 24 and allowing the bone paste to harden, thereby repairing or remodeling the bone.

44. An alginate-fibrin microbead, wherein said microbead comprises alginate and fibrinogen.

45. The alginate-fibrin microbead of claim 44, wherein the alginate-fibrin microbead comprises a fibrinogen mass fraction of from about 0.05% to about 1%.

46. The alginate-fibrin microbead of claim 44, wherein the alginate-fibrin microbead comprises a fibrinogen mass fraction of about 0.12%.

47. The alginate-fibrin microbead of any one of claims 44-46, wherein the microbead has an average diameter of less than about 2 millimeters.
